SEQUENCE LISTING (1 sequence)

SEQ ID NO: 1 - DNA, 20 bp, Artificial sequence
  Synthetic
gcaagctgaccctgaagttc

(19) United States
(12) Patent Application Publication
(10) Pub. No.: US 2023/0381113 A1
(43) Pub. Date: Nov. 30, 2023

(54) POLYMERS AND NANOPARTICLE FORMULATIONS FOR SYSTEMIC NUCLEIC ACID DELIVERY

(71) Applicant: The Johns Hopkins University, Baltimore, MD (US)

(72) Inventors: Jordan J. Green, Baltimore, MD (US); Yuan Rui, Baltimore, MD (US); David Wilson, Baltimore, MD (US); Stephany Yi Tzeng, Baltimore, MD (US)

(21) Appl. No.: 18/246,063

(22) PCT Filed: Sep. 28, 2021
(86) PCT No.: PCT/US2021/052405

§ 371 Date: Mar. 21, 2023

Related U.S. Application Data
(60) Provisional application No. 63/084,173, filed on Sep. 28, 2020.

(51) Int. Cl. A61K 9/51; A61K 31/7105; C08F 299/02; G01N 33/50; C12N 15/113; G01N 21/64

(52) U.S. Cl. CPC A61K 9/5153; A61K 31/7105; C08F 299/024; G01N 33/5023; C12N 15/113; G01N 21/6428; C12N 2310/14; C12N 2320/32; G01N 2021/6439

(57) ABSTRACT

Polymers and nanoparticle formulations for systemic nucleic acid delivery, including mRNA, are disclosed. A bioassay for simultaneously measuring nanoparticle cell uptake and endosomal disruption also is disclosed.

### FEDERALLY SPONSORED RESEARCH OR DEVELOPMENT

This invention was made with government support under EY031097, EB028239, and CA228133 awarded by the National Institutes of Health. The government has certain rights in the invention.

### BACKGROUND

Cytosolic delivery of nucleic acids via nanoparticle vectors necessitates endosomal disruption and escape for effective delivery. This process remains poorly understood and has been demonstrated to be one of the primary barriers to effective transfection using non-viral vectors for nucleic acid delivery with only an estimated 1-2% of internalized siRNA delivered with lipid nanoparticles effectively reaching the cytosol. Gilleron et al. (2013). Effective delivery of larger nucleic acid cargoes, including mRNA and plasmid DNA, remain even less well understood but are of critical interest to the field.

### SUMMARY

In some aspects, the presently disclosed subject matter provides a composition comprising a compound of formula (I):

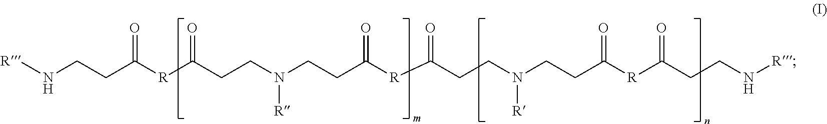

wherein: m and n are each integers from 1 to 10,000; R is derived from a linear diacrylate; R′ is derived from a hydrophobic amine; R″ is derived from a hydrophilic amine; and R′″ is an end-capping group.

In some aspects, the linear diacrylate comprises:

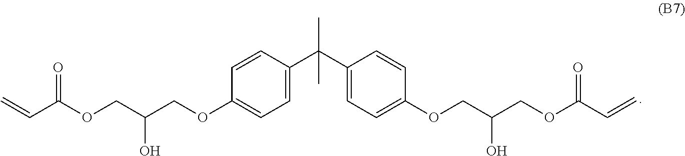 [SMILES: C=CC(=O)OCC(O)COc1ccc(C(C)(C)c2ccc(OCC(O)COC(=O)C=C)cc2)cc1]

In some aspects, the hydrophobic amine comprises:

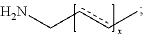

wherein x is an integer selected from 0, 1, 2, 3, 4, 5, 6, 7, 8, 9, 10, 11, 12, 13, 14, 15, 16, 17, 18, 19, and 20; and wherein

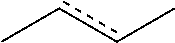

can be a single or double bond in one or more x repeating units.

In some aspects, the hydrophobic amine is selected from the group consisting of:

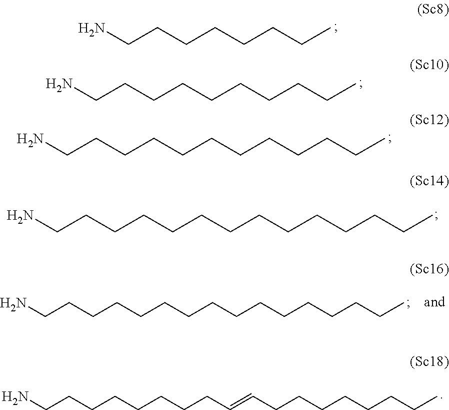

In some aspects, the hydrophilic amine comprises:

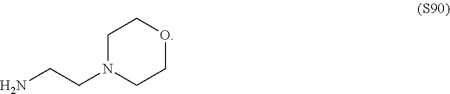

In some aspects, the end-capping group is selected from the group consisting of:

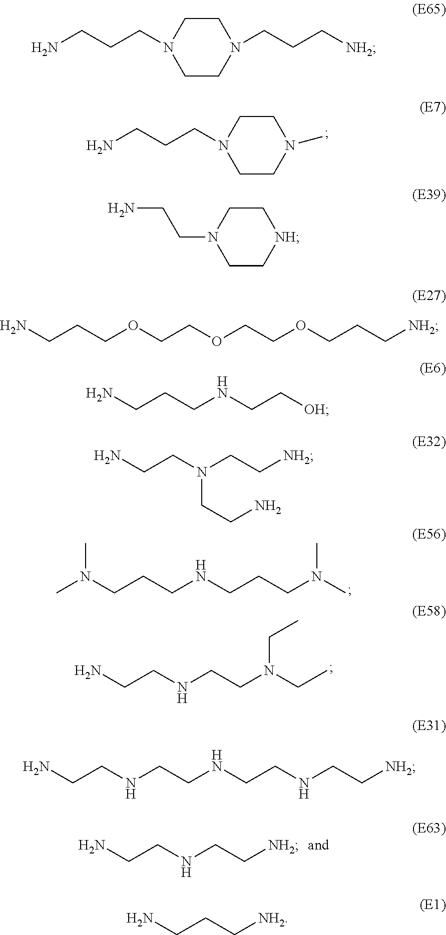

In some aspects, the composition further comprises one or more nucleic acids. In particular embodiments, the one or more nucleic acids is selected from the group consisting of mRNA, plasmid DNA, an oligonucleotide, a cyclic dinucleotide, other small nucleic acids, and combinations thereof.

In some aspects, the composition further comprises a PEG-lipid. In particular embodiments, the composition comprises about 0% to about 15% PEG-lipid.

In some aspects, the presently disclosed subject matter provides a formulation comprising the presently disclosed composition, wherein the formulation is one or more of frozen, lyophilized, or combined with one or more excipients to extend stability.

In other aspects, the presently disclosed subject matter provides a nanoparticle comprising the compositions described hereinabove.

In particular aspects, the nanoparticle targets a certain tissue.

In other aspects, the presently disclosed subject matter provides a method for systemic delivery of mRNA to a tissue, the method comprising administering a presently disclosed composition, a presently disclosed formulation, or a presently disclosed nanoparticle to the tissue. In certain embodiments, the tissue comprises tissue from an organ selected from the group consisting of lung, liver, kidney, heart, and spleen.

In other aspects, the presently disclosed subject matter provides a method for systemic deliver of mRNA to one or more immune cells, the method comprising administering a presently disclosed composition, a presently disclosed formulation, or a presently disclosed nanoparticle to the one or more immune cells.

In other aspects, the presently disclosed subject matter provides a method for treating a disease, condition, or disorder, the method comprising administering to a subject in need of treatment thereof a presently disclosed composition, a presently disclosed formulation, or a presently disclosed nanoparticle.

In other aspects, the presently disclosed subject matter provides a bioassay for simultaneously measuring nanoparticle cell uptake and endosomal disruption, the bioassay comprising: providing a nanoparticle comprising one or more fluorescent-labeled nucleic acids; incubating the nanoparticle with Gal8-mRuby+ cells; measuring nanoparticle uptake by quantifying fluorescent punta resulting from intracellular delivery of nanoparticles comprising the fluorescent-labeled nucleic acids; and measuring endosomal disruption by quantifying mRuby fluorescent puncta resulting from Gal8-mRuby clustering at damaged endosomal membranes.

In other aspects, the presently disclosed provides a kit comprising a presently disclosed composition, a presently disclosed formulation, or a presently disclosed nanoparticle.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 shows in vivo expression of polymer NPs delivering firefly luciferase mRNA following intravenous administration yields expression almost exclusively in animal lungs and spleen;

FIG. 2 shows that in untreated cells, Gal8-mRuby is found in a diffuse state through the cytosol;

FIG. 3A and FIG. 3B demonstrate that variation of polymer end-cap monomer has a strong influence on mRNA transfection efficacy in both (FIG. 3A) B16-F10 and (FIG. 3B) RAW264.7 cells;

FIG. 4A, FIG. 4B, and FIG. 4C show incorporation of eGFP expression from mRNA at 24 h post addition of nanoparticles for four-channel fluorescence assay to assess single-cell correlation between multiple variables. (FIG. 4A) Discrete counts of Gal8 and Cy5-NP puncta show high correlation indicating stochastic nature of escape; (FIG. 4B) High nanoparticle internalization is poorly correlated with gene expression at single cell level; and (FIG. 4C) High degree of endosomal disruption also poorly correlated with gene expression at single cell level;

FIG. 5A, FIG. 5B, and FIG. 5C demonstrate an image-based analysis of NP uptake and Gal8 endosomal disruption assay. (FIG. 5A) Assay overview: cells genetically encoding a Gal8-mRuby fusion fluorescence protein exhibited diffuse cytosolic mRuby signal in the absence of endosomal disruption. Endosomal disruption caused by NPs carrying Cy5-labeled nucleic acid NPs allow Gal8-mRuby to bind to intra-endosomal glycans, resulting in punctate fluorescent spots. (FIG. 5B) A typical field-of-view (taken from 80 per NP formulation) imaged by high-throughput fluorescence microscopy of B16-F10 murine melanoma cells after 6 h exposure to PBAE NPs carrying Cy5-mRNA. Cell identification was done using Hoechst 33342 staining of cell nuclei. Identification of Gal8-mRuby puncta and Cy5 puncta were used to quantify endosomal disruption and NP uptake, respectively. Scale bars=50 μm. (FIG. 5C) Representative distributions of the Gal8 puncta or Cy5 puncta count per cell obtained from image analysis data;

FIG. 6A, FIG. 6B, FIG. 6C, and FIG. 6D show the chemical structure and characterization of PBAE NPs. (FIG. 6A) PBAE synthesis via 2-step Michael Addition reactions for linear, end-capped polymers; (FIG. 6B) Structures of diacrylate (B), hydrophilic side chain (S), hydrophobic side chain (Sc), and endcap (E) monomers used in the synthesis of backbone hydrophobicity variation polymer series; (FIG. 6C) Representative TEM image of 7-90,c12-63, 50%-Sc12, mRNA NPs formulated at 60 w/w with 10% DMG-PEG2k and dialyzed into PBS. Scale bar=100 nm; (FIG. 6D) DLS measurements of z-average NP hydrodynamic diameter and zeta potential of 7-90,c12-63 50%-Sc12 NPs formed at 60 w/w and diluted into PBS. Data shown as mean+SD; n=3;

FIG. 7A, FIG. 7B, FIG. 7C, and FIG. 7D demonstrate validation of dual nanoparticle uptake/Gal8 endosomal disruption assay in PBAE nanoparticles and commercial reagents delivering different nucleic acid cargos to B16-F10 cells. (FIG. 7A) Heatmaps summarizing nanoparticle uptake, Gal8 endosomal disruption, and transfection efficacy data. Uptake and Gal8 data were obtained from high-throughput imaging analysis. Transfection efficacy was assessed by flow cytometry. For DNA and mRNA delivery, GFP fluorescence intensity for each formulation was normalized to the max fluorescence intensity across all treatment conditions. siRNA-mediated GFP knockdown was quantified by normalizing the percent GFP+ cells for siGFP treated wells to the corresponding formulation delivering scRNA control. Data presented as the mean of 4 replicate wells. Transfection efficacy of nanoparticles formed with PBAE polymers encapsulating all nucleic acid types was plotted against common predictor readouts, such as (FIG. 7B) various polymer characteristics, (FIG. 7C) nanoparticle properties, and (FIG. 7D) nanoparticle-cell interactions. Correlation significance was assessed for PBAE nanoparticles using Spearman's method, and data sets with statistically significant correlations were indicated with fitted lines. Data presented as mean±SD, n=4;

FIG. 8A, FIG. 8B, and FIG. 8C show the effects of polymer end-group structure on mRNA transfection efficacy in multiple cell lines. (FIG. 8A) NP uptake, Gal8 puncta count, and mRNA delivery efficacy of polymer end-group variation PBAE library on three different cell lines. Transfection efficacy was plotted against (FIG. 8B) Gal8 puncta count indicating endosomal disruption or (FIG. 8C) Cy5 puncta count indicating NP uptake. Data presented as mean±SD, n=4. Correlation significance in (FIG. 8B)-(FIG. 8C) were calculated using Spearman's method; a hyperbolic curve was fitted in (FIG. 8B) to indicate a statistically significant correlation;

FIG. 9A, FIG. 9B, FIG. 9C, FIG. 9D, FIG. 9E, FIG. 9F, FIG. 9G, and FIG. 9H show the in vivo validation of PEG-coated PBAE NPs delivering mRNA. (FIG. 9A) Schematic depicting the experimental workflow. PBAE polymers were dialyzed with fLuc mRNA and the PEG-lipid DMG-PEG2k in PBS to form PEG-coated mRNA NPs, which were administered intravenously. fLuc expression was assessed 24 h after NP injection. (FIG. 9B) Whole body bioluminescence was assessed for NPs formulated with PBAEs with differential backbone hydrophobicity and (FIG. 9C) representative IVIS images (N=3). (FIG. 9D) Whole body bioluminescence quantification for NPs formulated with 50%-Sc12 PBAEs with different end-groups (N=4). (FIG. 9E) In vivo transfection efficacy (from (FIG. 9B) and (FIG. 9D)) was plotted against in vitro transfection in B16 cells. Spearman's correlation was used to measure the strength of association between the two variables. Organ bioluminescence in the most highly expressing organs when varying (FIG. 9F) polymer backbone hydrophobicity (blue triangles below organ labels indicate increasing backbone hydrophobicity) and (FIG. 9G) polymer end-group structure. Statistical significance was determined using one-way ANOVA with Dunnett's post-hoc analysis comparing against the least hydrophobic polymer (0% Sc12) in (FIG. 9B) and (FIG. 9F) and against end-group E63 in (FIG. 9D) and (FIG. 9G). *P<0.05, **P<0.01, ***P<0.001, and ****P<0.0001. ns, not significant. (FIG. 9H) The organ targeting index as calculated by normalizing bioluminescent flux in each non-liver organ against that of the liver was calculated for the lungs and spleen in high-expressing polymers of the polymer end-group variation series (E7 was excluded due to minimal expression observed). Dotted black line indicates liver expression level. N=4. Data presented as mean±SD in all bar graphs;

FIG. 10A, FIG. 10B, FIG. 10C, and FIG. 10D demonstrate assessment of in vivo mRNA transfection in different cell types. (FIG. 10A) Experimental workflow: Ai9 mice were injected with PEG-coated 7-90-c12-63 80%-5c12 NPs encapsulating Cre mRNA and single cell level transfection could be detected by tdTomato expression, which was quantified 3 days post-injection using flow cytometry. (FIG. tdTomato+ cells as a percentage of the total cell population in each of several major organs. (FIG. 10C) tdTomato expression in the lungs in different cell types (tdTomato+ cells as a percentage of the overall population of each cell type). (FIG. Distribution of tdTomato+ cells across different cell types in liver, lungs, and spleen. N=3. Data presented as mean±SD in bar graphs;

FIG. 11A and FIG. 11B show time course optimization for dual NP uptake/Gal8 endosomal disruption assay. (FIG. 11A) Gal8 puncta count and (FIG. 11B) Cy5 puncta count for 7-90,c12-63, 50%-Sc12 NPs delivering various nucleic acid cargos to B16-F10 cells after different incubation times. Black arrow indicates the 6 h time point, which was chosen as the NP incubation time for this assay. Data presented as mean±SD, n=4;

FIG. 12A, FIG. 12B, and FIG. 12C show polymer and nanoparticle characteristics for the polymer backbone hydrophobicity variation series. (FIG. 12A) Z-average hydrodynamic diameter and (FIG. 12B) zeta potential for polymers encapsulating plasmid DNA, mRNA, siRNA, or polymer only nanoparticles (no nucleic acids). Data shown as mean±SD, n=3. (FIG. 12C) Polymer molecular weight as determined by GPC;

FIG. 13A, FIG. 13B, FIG. 13C, and FIG. 13D show the polymer effective pKa and pH titration curves. (FIG. 13A) Effective pKa in the physiologically relevant pH range for polymers in the backbone variation series. (FIG. 13B) Representative pH titration curves. (FIG. 13C) Normalized buffering capacity was calculated from pH titration data as Δ(OH)/Δ(pH) at each titration point (pH 5-8). (FIG. 13D) Effective pKa value of each polymer was determined as the pH point of the maximum normalized buffering capacity (indicated by red arrows in representative curves).

FIG. 14A, FIG. 14B, and FIG. 14C show RiboGreen nucleic acid binding data. (FIG. 14A) Tabulated polymer IC₅₀ of binding for polymers in the backbone variation series assessed with plasmid DNA, mRNA, and siRNA. (FIG. 14B) RiboGreen fluorescence quenching competitive binding curves for polymers in the alkyl chain length variation series. (FIG. 14C) Binding curves for polymers in the alkyl fraction variation series. Red line indicates 50% fluorescence quenching. Data shown as mean±SD, n=2;

FIG. 15A and FIG. 15B show correlations between in vitro transfection efficacy and polymer buffering capacity and hydrophobicity, respectively. Correlations between transfection efficacy and (FIG. 15A) polymer effective pKa in the physiological pH range or (FIG. 15B) predicted polymer Log P values of nanoparticles from backbone hydrophobicity variation polymers delivering different nucleic acid cargo (left) or end-group variation polymers delivering mRNA to different cell lines (right). Spearman's correlation was calculated to assess the strength of association between variable groups, and a line of best fit is shown for data sets with significant levels of correlation. Data shown as mean±SD, n=4;

FIG. 16A and FIG. 16B show IVIS images of BALB/c mice treated with NPs formulated with fLuc mRNA and select polymers from the backbone hydrophobicity variation series. (FIG. 16A) Whole-body, live animal bioluminescence imaging. (FIG. 16B) Bioluminescence imaging of select organs. Readings taken 24 h after NP injection. (n=3);

FIG. 17A and FIG. 17B show IVIS images of BALB/c mice treated with NPs formulated with fLuc mRNA and select polymers from the end-group variation series. (FIG. 17A) Whole-body, live animal bioluminescence imaging. (FIG. 17B) Bioluminescence imaging of select organs. Readings taken 24 h after NP injection. (n=4);

FIG. 18A, FIG. 18B, FIG. 18C, FIG. 18D, FIG. 18E, FIG. 18F, and FIG. 18G show the effect of PEG-coating and dialysis on mRNA transfection. (FIG. 18A) DLS NP measurements of dialyzed, PEG-coated PBAE mRNA NPs with increasing lipid-PEG content. (FIG. 18B) Transfection efficacy and (FIG. 18C) Cy5 and Gal8 puncta count for NPs with various combinations of PEG-coating and dialysis (assay performed using B16-F10 cells and delivering 50 ng mRNA per 96-well). Data presented as mean±SD, n=3. Statistical significance calculated using one-way ANOVA with Tukey's post-hoc analysis. *P<0.05, **P<0.01, and ***P<0.001. ns, not significant. IVIS bioluminescence imaging for (FIG. 18D) whole-body, live animals and (FIG. 18E) select organs in animals injected with dialyzed and PEG-coated or non-dialyzed and non-PEG-coated NPs. Readings taken 24 h after NP injection. Quantification of luminescence from (FIG. 18F) whole-body or (FIG. 18G) organ level images. Statistical significance calculated using Student's t-test with Welch's correction for (FIG. 18F) and 2-way ANOVA with Sidak's post-hoc analysis for (FIG. 18G). ***P<0.001. ns, not significant. (n=4); and

FIG. 19A and FIG. 19B show flow cytometry gating strategies to identify cell type expression in Ai9 mice. Representative flow cytometry histograms to identify (FIG. 19A) various immune cell populations (panel 1) or (FIG. 19B) immune and non-immune cells (panel 2) in the liver.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Polymers and Nanoparticle Formulations for Systemic Nucleic Acid Delivery

The presently disclosed subject matter, in part, solves the challenge of delivering mRNA and other nucleic acids safely and effectively to tissues and cells following systemic injection. It is a platform that can be used for many therapeutic purposes (cardiovascular disease, cancer, autoimmunity, and the like).

In some embodiments, the presently disclosed subject matter provides a composition comprising a compound of formula (I):

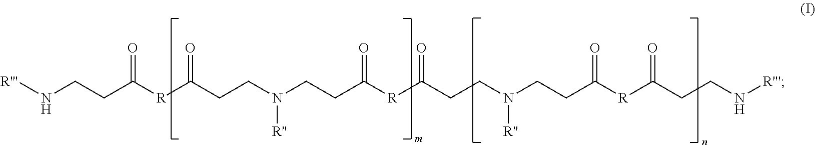

wherein: m and n are each integers from 1 to 10,000; R is derived from a linear diacrylate; R′ is derived from a hydrophobic amine; R″ is derived from a hydrophilic amine; and R′″ is an end-capping group.

In some embodiments, the linear diacrylate comprises:

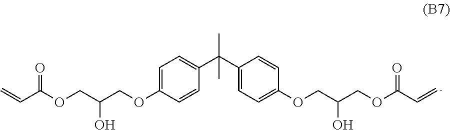 [SMILES: C=CC(=O)OCC(O)COc1ccc(C(C)(C)c2ccc(OCC(O)COC(=O)C=C)cc2)cc1]

In some embodiments, the hydrophobic amine comprises:

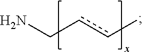

wherein x is an integer selected from 0, 1, 2, 3, 4, 5, 6, 7, 8, 9, 10, 11, 12, 13, 14, 15, 16, 17, 18, 19, and 20; and wherein

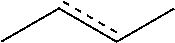

can ne a single or double bond in one or more x repeating units.

In some embodiments, the hydrophobic amine is selected from the group consisting of:

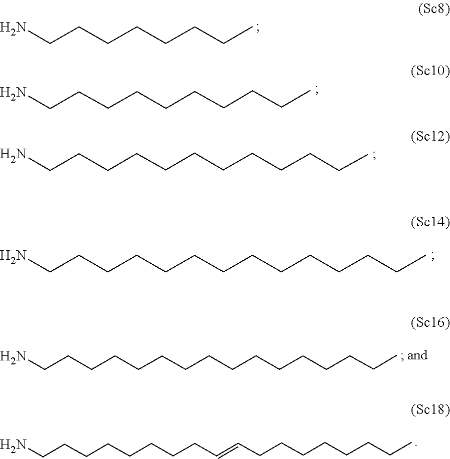

In some embodiments, the hydrophilic amine comprises:

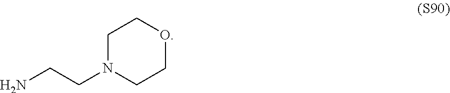

In some embodiments, the end-capping group is selected from the group consisting of:

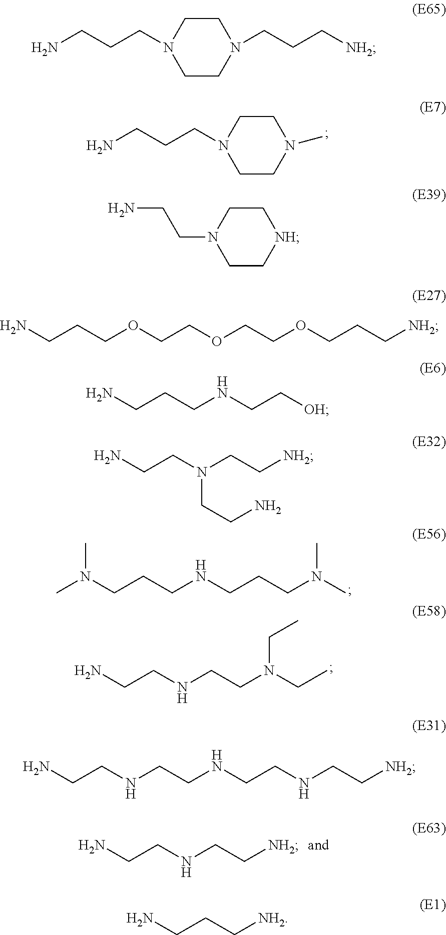

In particular embodiments, the end-capping group is:

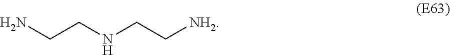

In some embodiments, the linear diacrylate is B7 and the hydrophobic amine is a blend of S90 and Sc12 and the end-capping group is selected from the group consisting of:

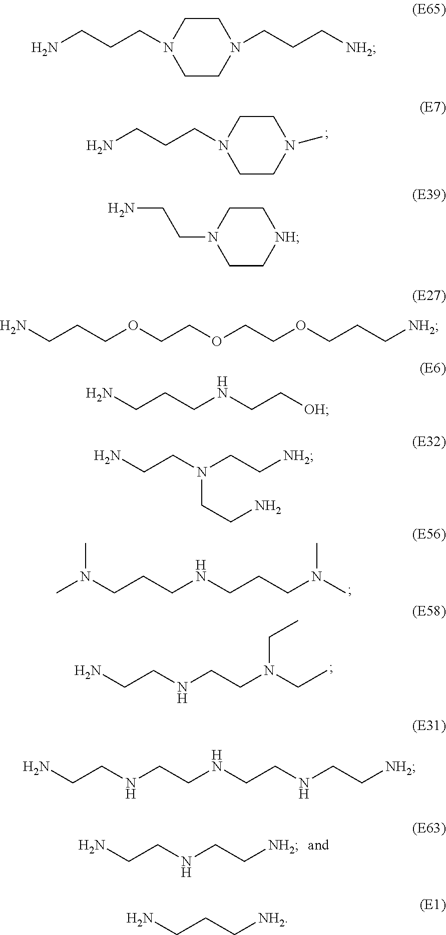

In some embodiments, the linear diacrylate is B7, the end-capping group is E63, the hydrophilic amine is S90, and the hydrophobic amine is selected from the group consisting of S8, S10, S12, S14, S16, and S18.

In some embodiments, at least one of S8, S10, S12, S14, S16, and S18 is present at a percentage ranging from about 15% to 80% relative to a percentage of S90, including about 15, 20, 25, 30, 35, 40, 45, 50, 55, 60, 65, 70, 75, and 80% relative to a percentage of S90.

In some embodiments, the composition further comprises one or more nucleic acids. In particular embodiments, the one or more nucleic acids is selected from the group consisting of mRNA, plasmid DNA, an oligonucleotide, a cyclic dinucleotide, other small nucleic acids, and combinations thereof.

In some embodiments, the composition further comprises a PEG-lipid. In particular embodiments, the composition comprises about 0% to about 15% PEG-lipid, including 0, 1, 2, 3, 4, 5, 6, 7, 8, 9, 10, 11, 12, 13, 14, and 15% PEG-lipid.

In particular embodiments, the end-capping group is selected from the group consisting of E63, E1, E58, E39, and E7.

In some embodiments, the presently disclosed subject matter provides a formulation comprising the presently disclosed composition, wherein the formulation is one or more of frozen, lyophilized, or combined with one or more excipients to extend stability.

For example, in some embodiments, the presently disclosed subject matter also includes a method of using and storing the polymers and particles described herein whereby a cryoprotectant (including, but not limited to, a sugar) is added to the polymer and/or particle solution and it is lyophilized and stored as a powder. Such a powder is designed to remain stable and be reconstituted easily with aqueous buffer as one skilled in the art could utilize. Moreover, freeze-dried nanoparticles typically are stable for up to two years when stored at room temperature, 4° C. or −20° C. In some embodiments, the composition is lyophilized, and reconstituted prior to administration to a subject, e.g. a patient.

Depending on the specific conditions being treated, the pharmaceutical composition may be formulated into liquid or solid dosage forms and administered systemically or locally. The pharmaceutical composition may be delivered, for example, in a timed- or sustained-low release form as is known to those skilled in the art. Techniques for formulation and administration may be found in “Remington: The Science and Practice of Pharmacy (20th ed.)” Lippincott, Williams & Wilkins (2000). Suitable routes may include oral, buccal, by inhalation spray, sublingual, ocular, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, intratumoral, intraocular (e.g., intravitreal) injections, or other modes of delivery.

While the form and/or route of administration can vary, in some embodiments the pharmaceutical composition is formulated for parenteral administration (e.g., by subcutaneous, intravenous, or intramuscular administration).

Formulations may optionally contain at least one particulate pharmaceutically acceptable carrier known to those of skill in the art. Examples of suitable pharmaceutical carriers include, but are not limited to, saccharides, including monosaccharides, disaccharides, polysaccharides and sugar alcohols such as arabinose, glucose, fructose, ribose, mannose, sucrose, trehalose, lactose, maltose, starches, dextran, mannitol or sorbitol.

Use of pharmaceutically acceptable inert carriers to formulate pharmaceutical compositions disclosed herein into dosages suitable for systemic administration is within the scope of the present invention. With proper choice of carrier and suitable manufacturing practice, the compositions of the present invention, in particular, those formulated as solutions, may be administered parenterally, such as by intravenous injection, or locally, such as intraocular injection. The pharmaceutical compositions can be formulated readily using pharmaceutically acceptable carriers well known in the art into dosages suitable for oral administration. Such carriers enable the pharmaceutical composition to be formulated as tablets, pills, capsules, liquids, gels, syrups, slurries, suspensions and the like, for oral ingestion by a subject (e.g., patient) to be treated.

For injection, pharmaceutical compositions of the present invention may be formulated and diluted in aqueous solutions, such as in physiologically compatible buffers such as Hanks' solution, Ringer's solution, or physiological saline buffer.

In certain embodiments, the presently disclosed subject matter provides a pharmaceutical formulation of comprising the presently disclosed compositions in a pharmaceutically acceptable carrier. As used herein, “pharmaceutically acceptable carrier” is intended to include, but is not limited to, water, saline, dextrose solutions, human serum albumin, liposomes, hydrogels, microparticles and nanoparticles. The use of such media and agents for pharmaceutically active compositions is well known in the art.

In other embodiments, the presently disclosed subject matter provides a nanoparticle comprising the compositions described hereinabove. In embodiments, the particle has at least one dimension in the range of about 50 nm to about 1,000 nm, or, in embodiments, from about 50 to about 500 nm. Exemplary particles may have an average size (e.g., average diameter) of about 50, about 75, about 100, about 125, about 150, about 200, about 250, about 300, about 400 or about 500 nm. In some embodiments, the nanoparticle has an average diameter of from about 50 nm to about 500 nm, from about 50 nm to about 300 nm, or from about 50 nm to about 200 nm, or from about 50 nm to about 150 nm, or from about 70 to 100 nm. In embodiments, the nanoparticle has an average diameter of from about 200 nm to about 500 nm. In embodiments, the nanoparticle has at least one dimension, e.g., average diameter, of about 50 to about 100 nm. Nanoparticles are usually desirable for in vivo applications. For example, a nanoparticle of less than about 200 nm will better distribute to target tissues in vivo.

In particular embodiments, the nanoparticle targets a certain tissue.

In some embodiments, the nanoparticle comprises greater than about 50% of a dry particle mass.

In other embodiments, the presently disclosed subject matter provides a method for systemic delivery of mRNA to a tissue, the method comprising administering a presently disclosed composition, a presently disclosed formulation, or a presently disclosed nanoparticle to the tissue. In certain embodiments, the tissue comprises tissue from an organ selected from the group consisting of lung, liver, kidney, heart, and spleen.

In other embodiments, the presently disclosed subject matter provides a method for systemic deliver of mRNA to one or more immune cells, the method comprising administering a presently disclosed composition, a presently disclosed formulation, or a presently disclosed nanoparticle to the one or more immune cells.

In other embodiments, the presently disclosed subject matter provides a method for treating a disease, condition, or disorder, the method comprising administering to a subject in need of treatment thereof a presently disclosed composition, a presently disclosed formulation, or a presently disclosed nanoparticle.

In certain embodiments, the composition or nanoparticle comprises one or more of mRNA, plasmid DNA, an oligonucleotide, a cyclic dinucleotide, other small nucleic acids, and combinations thereof.

In particular embodiments, the administration comprises an intravenous injection.

In other embodiments, the presently disclosed subject matter provides a bioassay for simultaneously measuring nanoparticle cell uptake and endosomal disruption, the bioassay comprising: providing a nanoparticle comprising one or more fluorescent-labeled nucleic acids; incubating the nanoparticle with Gal8-mRuby+ cells; measuring nanoparticle uptake by quantifying fluorescent punta resulting from intracellular delivery of nanoparticles comprising the fluorescent-labeled nucleic acids; and measuring endosomal disruption by quantifying mRuby fluorescent puncta resulting from Gal8-mRuby clustering at damaged endosomal membranes.

In certain embodiments, the fluorescent punta are quantified via images obtained by wide-field, epifluorescence microscopy.

In other embodiments, the presently disclosed provides a kit comprising a presently disclosed composition or a presently disclosed nanoparticle.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

### II. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### A Genetically Encoded Sensor of Endosomal Disruption Demonstrates Efficient Entry to the Cytosol Using Polymeric Nanoparticles for mRNA Delivery

### 1.1 Overview

Poly(beta-amino ester) (PBAE)-based nanoparticles were used to deliver both DNA and RNA. Wittrup et al. (2015) and Kilchrist et al. (2019) previously identified the cytosolic protein galectin-8 (Gal8) as a carbohydrate recognizing protein critically involved in formation of autophagosomes following endosomal disruption. In this example, multiple cell lines were engineered to express a Gal8-mRuby fusion protein construct. Wittrup et al. (2015) and Kilchrist et al. (2019). Following endosomal disruption, Gal8 clusters around disrupted sections of endosomal membrane, binding to lectins found on the outer leaflet of the plasma membrane. Expression of the Gal8-mRuby fusion protein construct enabled image-based assessment and quantification of bright Gal8-mRuby puncta that form in response to endosomal disruption in a high-throughput manner facilitated by automated 20×-widefield image acquisition.

Using this assay, multiple structure-function relationships between the polymeric structure of poly(beta-amino ester)s (PBAEs) and single cell levels of endosomal disruption, nucleic acid uptake and functional cytosolic delivery were probed. More particularly, the influence of lipophilicity (via inclusion of amino-alkanes), cationicity and branching in PBAE structure was investigated to demonstrate that lipophilicity does not influence endosomal disruption efficiency, whereas branching and cationicity were positively correlated with endosomal disruption efficiency with up to 50% of internalized nanoparticles demonstrating endosomal disruption.

The kinetics of endosomal disruption with these nanomaterials was further probed, demonstrating that PBAEs enable rapid escape following internalization from early endosomes with multiple separate disruption events occurring for each transfected cell. These results also indicate that the most effective materials for mRNA delivery may delay the formation of membrane enclosed autophagosomes that limits functional nucleic acid escape from disrupted vesicles in contrast to canonical polycations, such as polyethyleneimine, which appears to disrupt many late-endosomes/lysosomes and has a very short window of escape for functional cytosolic delivery.

In summary, this assay and results suggest a path forward to engineering nanomaterials that are more efficient for endosomal escape, potentially improving functional cytosolic delivery or mRNA both in vitro and in vivo.

This example examines, in part, whether endosomal disruption is influenced by particular nanoparticle features, including polymer structure (such as, hydrophobicity) and nucleic acid content (siRNA vs mRNA vs plasmid vs empty) and whether differences in endosomal disruption are responsible for differences between ease of transfection in different cell types.

### 1.2 Nanoparticle Formation and In Vivo Expression

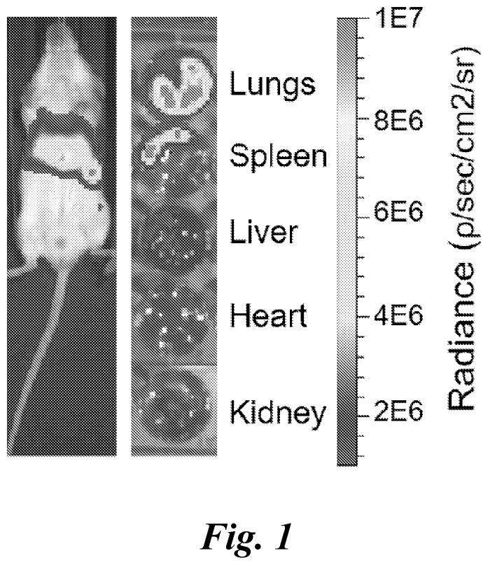

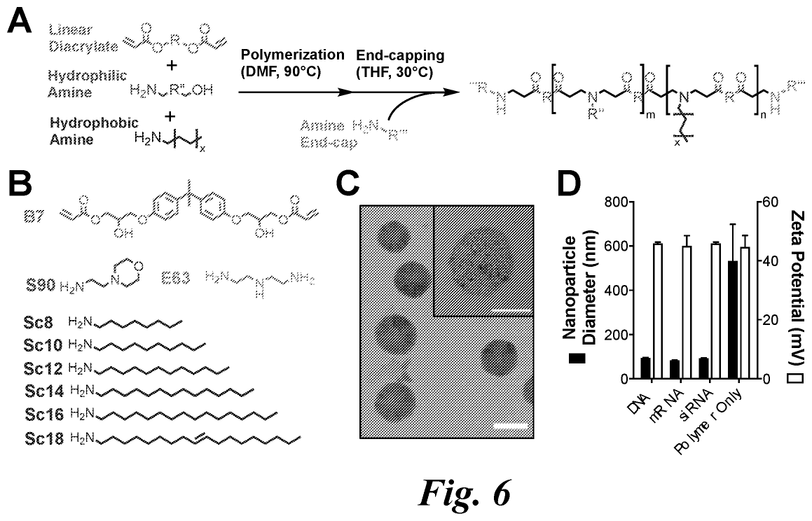

Referring now to FIG. 6C and FIG. 1 are shown TEM of dialyzed PBAE-based polymer NPs. FIG. 1B shows in vivo expression polymer NPs delivering firefly luciferase mRNA following intravenous administration yields expression almost exclusively in animal lungs and spleen.

### 1.3 Gal8 Assay Establishment

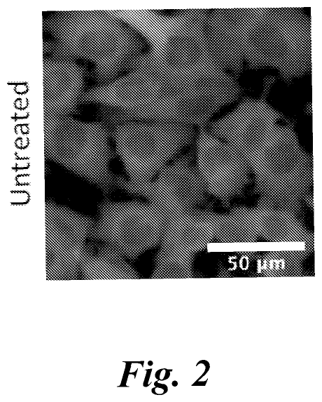

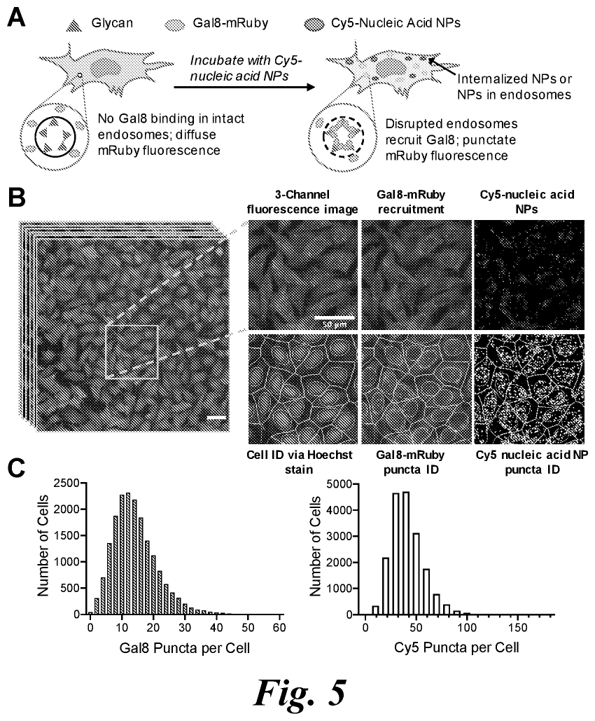

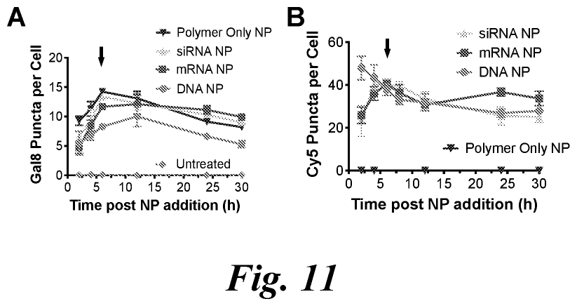

Referring now to FIG. 2 and FIGS. 5A-5B, and FIG. 11A-11B, FIG. 5A shows that cell lines expressing a galectin-8 (Gal8) fusion protein with mRuby3 were transformed using a piggybac transposon construct to create a genetically encoded reporter for endosomal disruption as reported by Kilchrist et al. 2019. Gal8-mRuby (shown in green for visibility) binds to lectins found on glycosylated extracellular membrane proteins. FIG. 2 shows that in untreated cells, Gal8-mRuby is found in a diffuse state through the cytosol. FIG. 5B demonstrates that nanoparticles that induce endosomal disruption led to the formation of Gal8-mRuby fluorescence puncta that can be counted via image analysis. Cy5-labeled nucleic acid puncta for nucleic acid internalization also can be counted. FIG. 11A shows that Gal8 puncta for endosomal disruption peaks at approximately 6 h and persists over 24 h, slightly decreasing due to mergers between autophagosomes. Kilchrist et al. 2019. FIG. 11B shows that nanoparticle uptake peaks at approximately 4 h and remains nearly constant.

### 1.4 Length of Alkyl Side-Chain

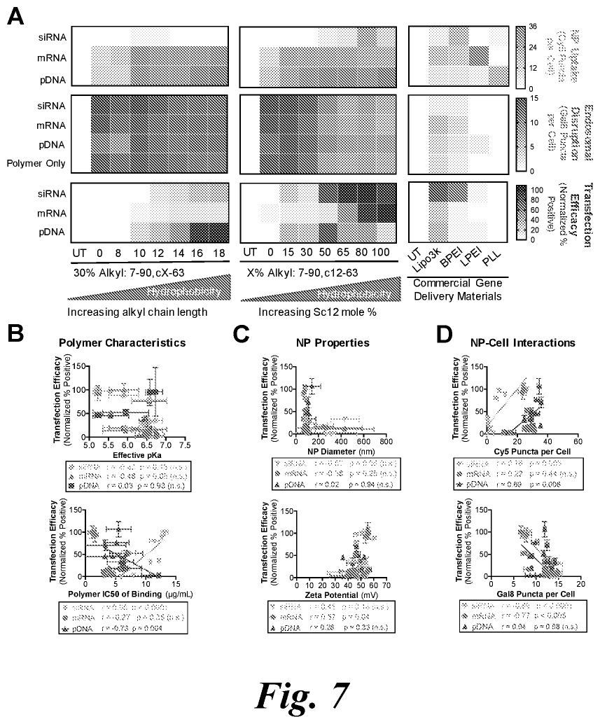

Referring now to FIG. 7A, FIG. 7A (left panel, bottom panel) shows the transfection efficacy in B16-F10 cells using the same nanoparticles for siRNA, mRNA or plasmid DNA while varying the amino-alkane carbon chain length. Increases in the alkyl-chain length improved transfection efficacy, with the greatest improvements in transfection noted for siRNA delivery. FIG. 7A (left panel, upper panel) shows that the number of Cy5 NP puncta measured via image analysis demonstrates improved cell uptake when amino-alkyl monomers have at least 10 carbon atoms. FIG. 7A (left panel, middle panel) shows the number of Gal8 endosomal disruption puncta detectable via image analysis, demonstrating that inclusion of amino-alkane side-chains of any length did not influence endosomal disruption.

### 1.5 Alkyl Mole Fraction (%)

Referring once again to FIG. 7A, using the same overall polymer structure varying the percent of amino monomers that are the amino-alkane between 0-100% demonstrates the influence of transfection efficacy in B16-F10 cells using the same nanoparticles for siRNA, mRNA or pDNA while varying amino-alkane mole fraction from 0% to 100% (FIG. 7A, middle panel, bottom panel). Higher alkyl-mole fractions correlated with better transfection, particularly for siRNA. FIG. 7A (middle panel, top panel) shows that Cy5-labeled nucleic acid uptake by nanoparticles demonstrates that higher alkyl mole % yields improved cell uptake (particularly for siRNA). FIG. 7A (middle panel, middle panel) shows that endosomal disruption decreases with increasing alkyl mole %.

### 1.6 End-Cap Monomer

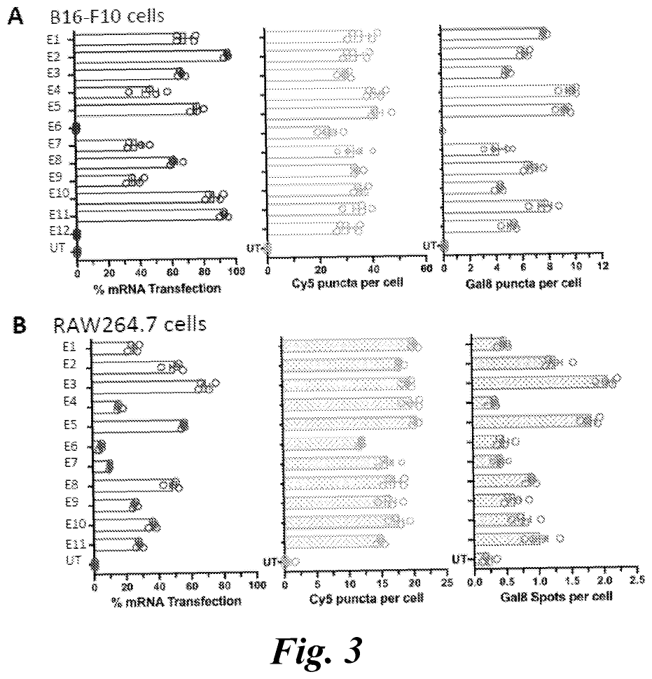

Referring now to FIG. 3A-3B, it is shown that variation of the polymer end-cap monomer has a strong influence on mRNA transfection efficacy in both B16-F10 (FIG. 3A) and RAW264.7 cells (FIG. 3B). These data indicate that the end-cap monomer strongly influences Gal8 disruption while minimally affecting degree of nanoparticle uptake in either cell line, i.e., E6 end-capped polymers form NPs and deliver mRNA to endosomes, but fail to escape and enable translation of mRNA; Gal8 monitored endosomal disruption (Gal8 puncta per cell) predicts transfection efficacy more effectively than nanoparticle internalization (Cy5 puncta); B16-F10 cancer cells are generally easier to transfect than RAW264.7 “macrophages” that are phagocytic in nature; B16-F10 cells are much larger overall and internalize a greater number of nanoparticles, but the number of discrete NP internalization events between cell types <2× higher for B16. In contrast, the number of Gal8 disruption events is approximately 5× higher in B16 cells, indicating that nanoparticles internalized by B16-F10 cells induce endosomal disruption at rates approximately 2.5× higher than RAW264.7 cells.

### 1.7 Commercial Gene Delivery Vectors

Referring now to FIG. 7A (right panel), commercial and canonical materials for delivery in vitro including Lipofectamine 3000, branched/linear polyethylenimine and poly-L-lysine are shown. FIG. 7A (right panel, bottom panel) shows the transfection efficacy for B16-F10 cells with mRNA. FIG. 7A (right panel, top panel) demonstrates that cell uptake is not predictive of degree of transfection. FIG. 7A (right panel, middle panel) shows that Gal8 disruption is much lower than with PBAEs and correlates strongly with transfection efficacy indicating endosomal disruption is a primary barrier to effective intracellular delivery.

### 1.8 Single-Cell Endosomal Disruption, Uptake and Expression

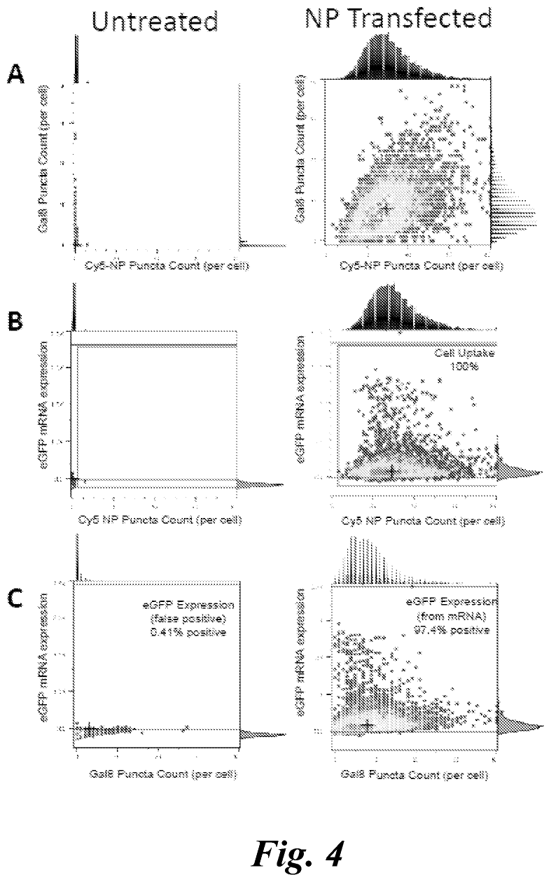

Referring now to FIG. 4A-FIG. 4C, illustrates incorporation of eGFP expression from mRNA at 24 h post addition of nanoparticles for a four-channel fluorescence assay to assess single-cell correlation between multiple variables. FIG. 4A demonstrates that discrete counts of Gal8 and Cy5-NP puncta show high correlation indicating stochastic nature of escape; FIG. 4B shows that high nanoparticle internalization is poorly correlated with gene expression at single cell level; and FIG. 4C shows that a high degree of endosomal disruption also poorly correlated with gene expression at single cell level.

### 1.9 SUMMARY

Inclusion of alkyl side-chains primarily improves polymer efficacy by improving cell uptake of nucleic acids, while slightly reducing endosomal disruption efficacy. End-cap monomers can strongly influence endosomal disruption, while minimally affecting cell uptake. Endosomal disruption is likely the primary barrier to transfection in a cell-type dependent manner. Single-cell level correlation between uptake, endosomal disruption and mRNA expression demonstrates a correlation between individual cell nanoparticle internalizations and endosomal disruption and cell internalizing moderate numbers of nanoparticles and having moderate degree of Gal8 disruption events have highest mRNA gene expression.

### Example 2

### Modifications to Poly(Beta-Amino Ester) Structure Influence Endosomal Escape, Cellular Uptake and Delivery of mRNA Nanoparticles In Vitro and In Vivo

### 2.1 Overview

Nanoparticle-based mRNA therapeutics hold great promise for the treatment of a variety of diseases. Cellular internalization and endosomal escape, however, remain key barriers in functional, cytosolic mRNA delivery. To facilitate in vitro identification of potent mRNA nanoparticle formulations, the presently disclosed subject matter provides a dual nanoparticle uptake and endosomal disruption assay using high throughput and high content image-based screening. Using a genetically encoded Galectin 8 fluorescent fusion protein sensor (Gal8-mRuby), endosomal disruption could be detected 6 hours after nanoparticle treatment via Gal8-mRuby clustering on damaged endosomal membranes. Simultaneously, nucleic acid endocytosis was quantified using fluorescently-tagged mRNA. An array of biodegradable poly(beta-amino ester)s, as well as Lipofectamine and polyethyleneimine (PEI), were used to demonstrate that this assay has higher predictive capacity for in vitro mRNA delivery compared to conventional polymer and nanoparticle physiochemical characteristics. Representative nanoparticle formulations enabled safe and efficacious mRNA expression in multiple tissues following intravenous injection, demonstrating that this in vitro screening method also is predictive of in vivo performance. Efficacious non-viral systemic delivery of mRNA with biodegradable particles opens up new avenues for genetic medicine and human health.

### 2.2 Background

Recent advances in the synthesis of in vitro transcribed (IVT) mRNA, Karikó et al., 2008; Thess et al., 2015, has spurred a vast amount of research into mRNA-based gene therapies including the development of next generation vaccines. Corbett et al., 2020. Compared to their plasmid DNA counterparts, mRNA offers safer and more controlled gene expression by virtually eliminating the risk for integration into the host genome. Pardi et al, 2018. mRNA delivery also could lead to more potent expression in cell populations that are largely refractory to DNA transfection, such as T cells, which have been shown to mount immune responses against foreign cytosolic DNA. Mandal et al., 2014; Monroe et al., 2014. Due to their size and hydrophilicity, however, mRNA molecules are membrane-impermeable, making safe and efficient cytosolic mRNA delivery a major obstacle to their clinical utility.

Non-viral nanoparticle (NP) formulations have emerged as promising mRNA delivery vehicles. Many lipid-based, Sabnis et al., 2018, and several polymeric, Patel et al., 2019, mRNA NP systems have recently been reported for protein replacement, Cheng et al., 2018; Cao et al, 2019, immune modulation, Billingsley et al., 2020; Miao et al., 2019, and gene editing applications. Liu et al., 2019; Miller et al., 2017. To fully realize the promise of mRNA therapeutics, NP systems must be engineered to overcome intracellular barriers, such as cellular internalization and escape from endosomal sequestration. Rui et al., 2019. A study of lipid NPs encapsulating siRNA showed that only an estimated 1-2%, Gilleron et al., 2013, of internalized siRNA reaches the cytosol, highlighting the need for improved nanomaterials, as well as quantitative high-throughput in vitro assays that can measure NP performance at key delivery bottlenecks and improve NP design.

Several image-based methods for quantifying the ability of NPs to overcome endosomal entrapment have been reported. The most common method is assessing the lack of co-localization of fluorescently labeled NPs with the pH-sensitive Lysotracker dye, Tamura et al, 2009; Akita et al., 2010, which selectively accumulates in the acidic environment of endosomes. This approach is easy to use and applicable to a wide variety of materials, but only provides an indirect assessment, as it does not indicate effective endosomal escape or disruption. Transmission electron microscopy (TEM) imaging is another widely accepted method for confirming endosomal disruption and escape. Gilleron et al., 2013; Kilchrist et al., 2016. This method, however, is not amenable to high-throughput analysis, cannot be done on living cells, and requires electron-dense labels, such as gold NPs, which could alter the properties of the native NP system. More recently, several groups have reported the use of advanced imaging approaches, such as high-dynamic-range confocal microscopy, Wittrup et al., 2015, or super-resolution stochastic optical reconstruction microscopy (STORM), Wojnilowicz et al., 2019, which have yielded important mechanistic data for the intracellular fate of the materials being studied, but lack the high-throughput screening capacity required to evaluate arrays of nanomaterials.

### 2.3 Scope

In this example, Galectin 8 (Gal8) tracking was used for high-throughput image-based quantification of endosomal disruption. Gal8 is a β-galactoside carbohydrate-binding protein that selectively binds to glycans found on the inner leaflet of endosomal membranes. Hadari et al., 1995; Thurston et al., 2012.

Using cells genetically engineered to constitutively express a Gal8-mRuby fusion protein, the endosomal disruption capabilities of nanocarriers were characterized by quantifying the fluorescent puncta that formed following Gal8-mRuby clustering on damaged endosomal membranes, building upon the Gal8 recruitment assay using PEG-(DMAEMA-co-BMA) siRNA NPs by Kilchrist et al., 2019. This approach was adapted to a high-throughput, widefield imaging assay to simultaneously study how cellular internalization and endosomal disruption correlated with nucleic acid delivery efficacy of biodegradable poly(beta-amino ester)s (PBAEs) and other common materials for nucleic acid delivery.

For PBAEs specifically, polymer backbone hydrophobicity, as well as polymer end-cap structure, were systematically varied to probe structure-function relationships. The predictive capacity of this dual cellular uptake and endosomal disruption assay was compared to that of several polymer and NP physiochemical properties, such as polymer nucleic acid binding strength, pH buffering capacity, predicted Log P value, NP hydrodynamic diameter, and zeta potential. The effects of nucleic acid cargo type, as well as cell type for in vitro transfection, were investigated.

In total, a library of 22 PBAEs with unique chemical structures was screened, as well as widely-used commercially-available transfection reagents, such as Lipofectamine™ 3000, polyethyleneimine (PEI), and poly-L-lysine (PLL). Finally, whether the presently disclosed in vitro screening assays correlated with systemic in vivo delivery efficacy of polymeric NPs encapsulating mRNA upon tail-vein injection in mice was examined. The data presented here demonstrate the robustness of this image-based dual NP uptake and endosomal disruption NP screening system across a broad range of materials for mRNA delivery efficacy in vitro, as well as in vivo. Such a quantitative, high-throughput screening platform with high predictive capacity for delivery efficacy has important implications for the standardization of the optimization and testing of novel materials for non-viral gene delivery and genetic medicine.

### 2.4 Results

### 2.4.1 High-Content Imaging of NP Uptake and Endosomal Disruption

B16-F10 murine melanoma cells were engineered to genetically encode a Gal8-mRuby endosomal disruption sensor to facilitate simultaneous characterization of NP uptake and endosomal disruption. NP uptake was measured by quantifying Cy5 puncta resulting from intracellular delivery of NPs carrying Cy5-labeled nucleic acids; endosomal disruption was measured by quantifying mRuby puncta resulting from Gal8-mRuby clustering at damaged endosomal membranes (FIG. 5A). This dual NP uptake and endosomal disruption assay was performed in a high-throughput manner using a CellInsight CX7 LZR high content imager capturing 20 fields of view per well of a 96-well plate at 20× magnification. An image analysis algorithm was then optimized and used to identify cells by extrapolating the cell body surrounding Hoechst 33342-stained cell nuclei and provide puncta counts per cell (FIG. 5B). On average, intracellular puncta count was collected for over 15,000 cells per NP formulation.

To identify the optimal time point to conduct the assay, a time course experiment was performed in which B16-mRuby-Gal8 cells were incubated with PBAE NPs for up to 30 h and imaged at select time points. It was found that the Cy5 and Gal8 puncta counts both peaked at 6 h post-transfection for most nucleic acid cargo types and generally decreased thereafter (FIG. 11), guiding us to perform this assay at 6 h for all remaining experiments. The decreases in Gal8 and Cy5 puncta over time are consistent with expected autophagy timelines for damaged endocytic vesicles. Wittrup et al., 2015.

### 2.4.2 Effects of PBAE Backbone Hydrophobicity

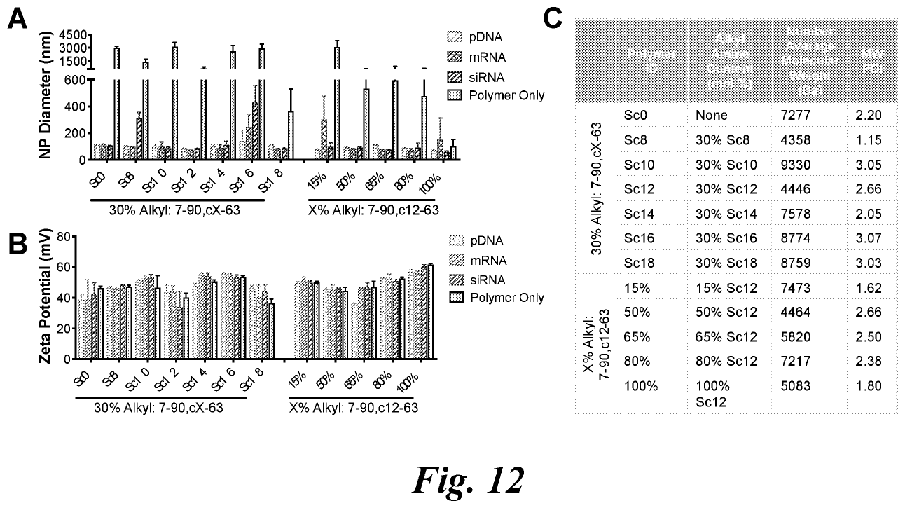

Two series of PBAE polymers with varying hydrophobic monomer content were synthesized to investigate the effects of polymer backbone hydrophobicity on NP uptake, endosomal disruption, and transfection capabilities. These lipophilic PBAE terpolymers consisted of a linear diacrylate (B7) copolymerized with a hydrophilic amine (S90) and a hydrophobic amine (ScX) synthesized via Michael Addition reactions (FIG. 6A). Polymer hydrophobicity was varied in one series by incorporating hydrophobic amines of varying lipid tail length at 30 mol % and in a second series by varying the molar content of the Sc12 monomer. Polymers in both series were then end-capped with monomer E63 to create PBAE quadpolymers, and molecular weight was found to be in the range of 4-10 kDa. All polymers were found to rapidly self-assemble into NPs with plasmid DNA, mRNA, and siRNA after simple pipette-mixing in aqueous buffer. NPs encapsulating nucleic acid cargo were 100 nm-400 nm in diameter with positive zeta potential in the range of 30-60 mV (FIG. 12).

Next, NP uptake, endosomal disruption, and gene delivery efficacy were assessed. In both PBAE polymer series, increasing polymer backbone hydrophobicity generally increased nucleic acid uptake and transfection in all three nucleic acid modalities (FIG. 7A). The opposite was true for Gal8 endosomal disruption, where the polymer containing 100% Sc12 (most hydrophobic) resulted in half of the Gal8-mRuby puncta count compared to the polymer containing 0% Sc12 (least hydrophobic). Commercially available gene delivery materials were used to provide a benchmark for the bioassays. Of the five commercially available materials tested, Lipofectamine™ 3000 enabled the highest transfection across all nucleic acid types, followed by 25 kD branched PEI. Transfection by siRNA NPs was assessed by siRNA-mediated GFP knockdown in cells engineered to be GFP+, while transfection by DNA and mRNA NPs was assessed by GFP expression resulting from functional delivery of DNA or mRNA encoding the GFP gene in non-GFP+ cells. Transfection with these commercially available materials correlated positively with endosomal disruption (Spearman's coefficient of 0.68), and no significant correlation with NP uptake was observed. The Gal8 puncta counts for these materials were much lower than those achieved by PBAE NPs even when transfection efficacy was similar, suggesting that the two classes of materials utilize different mechanisms to enable endosomal disruption.

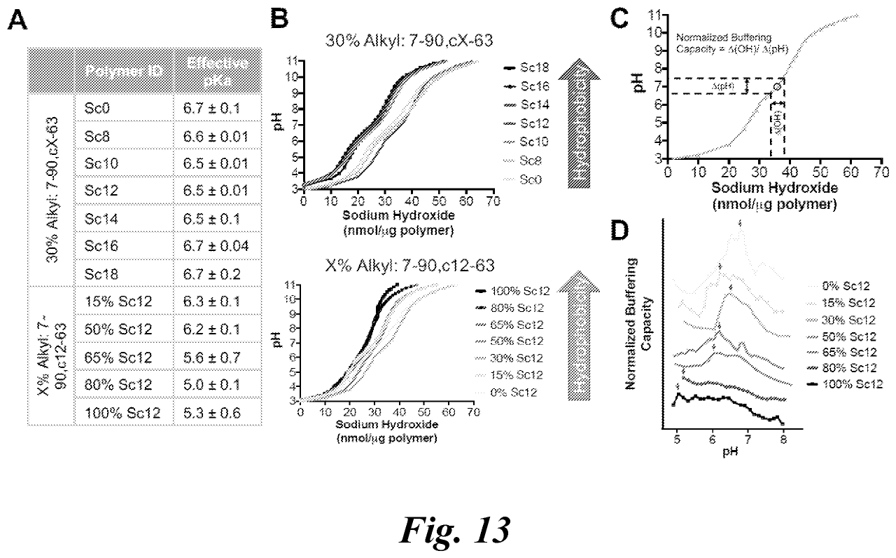

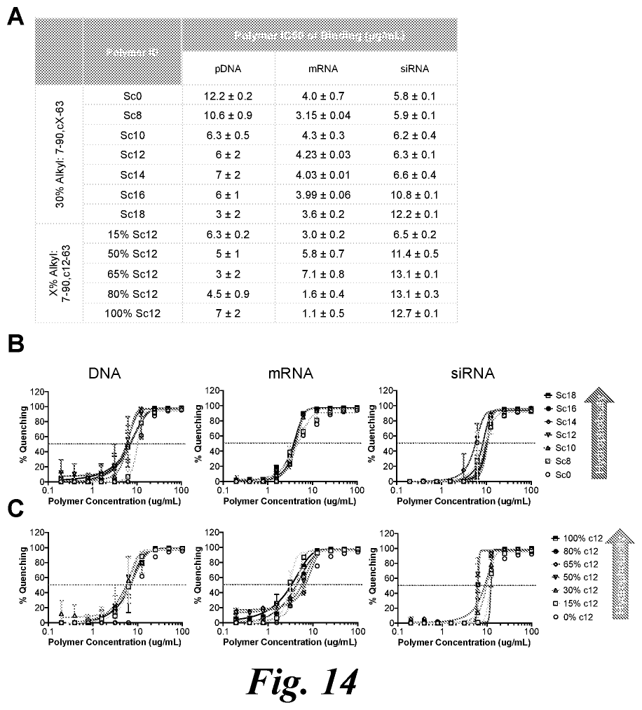

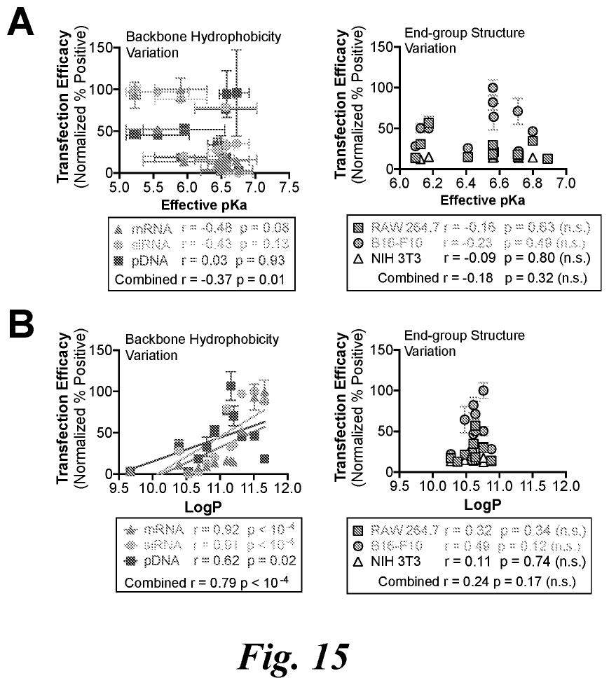

The predictive capacity of various polymer and NP properties on transfection efficacy was further assessed. The polymer IC₅₀ of nucleic acid binding, with larger values indicating weaker nucleic acid binding affinity, correlated negatively with DNA transfection but positively with siRNA knockdown. This observation may be due to the different intracellular sites of action for each nucleic acid. Plasmid DNA needs to reach the nucleus and strong initial binding could facilitate nuclear trafficking and maximize likelihood of transfection in each cell. On the other hand, siRNA needs to only be released to the cytosol to be active, and thus weaker polymer-nucleic acid binding could enable quicker and more effective cargo release and activity. mRNA transfection was not observed to correlate significantly with nucleic acid binding affinity in these experiments (FIG. 7B and FIG. 14). Standard biophysical characterization measurements of NP size and zeta potential showed no significant correlations with transfection efficiency (FIG. 3C). The NP-cell interactions quantified by the presently disclosed high-throughput and high-content imaging-based assay showed that PBAE transfection generally correlated positively with NP uptake and negatively with Gal8 endosomal disruption (FIG. 7D). The negative correlation between transfection and endosomal disruption levels in this series of PBAE NPs was surprising, although, even at their lowest, the endosomal disruption levels achieved with the PBAE NPs were significantly higher than those induced by the commercial gene delivery materials. Thus, all PBAE NPs evaluated may be above a critical threshold of endosomal disruption capacity necessary to enable functional nucleic acid delivery that is at least equal to or greater than the endosomal disruption capacity achieved by commercial gene delivery materials. The data indicated that in these experiments endosomal disruption was not a major transfection bottleneck for PBAEs. Interestingly, empty PBAE polymeric NPs in the absence of nucleic acids resulted in equivalent levels of endosomal disruption as NPs loaded with nucleic acids (FIG. 7A). This observation may explain why certain PBAE NP formulations less effective at transfection nonetheless exhibited high levels of endosomal disruption as these polymers may have formed a larger fraction of empty NPs. Such empty PBAE NPs could lead to a high Gal8 puncta count, indicating endosomal disruption, but would do so in a non-productive manner as no nucleic acids would be delivered to the cytosol. Interestingly, PBAE transfection with this series of polymers also did not correlate significantly with the polymers' effective pKa, as quantified in the physiologically relevant pH range (FIG. 7B, FIG. 13, and FIG. 15A), which also reinforces that endosomal disruption is not a rate-limiting step for these PBAE NPs to achieve intracellular delivery under these conditions. Other polymer properties, such as the predicted Log P value, which is a measure of polymer hydrophobicity, showed strong positive correlations with transfection for all three cargo types (FIG. 15B), further confirming the hypothesis that increased backbone hydrophobicity improves polymeric gene delivery efficacy.

### 2.4.3 Effects of Polymer End-Groups

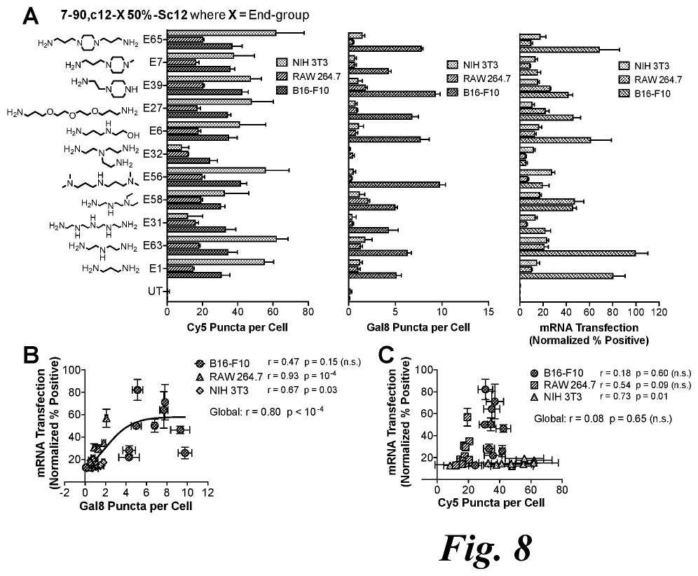

Next, the effects of polymer end-group structure on NP uptake and endosomal disruption were investigated by synthesizing an end-group variation polymer series using a moderately hydrophobic polymer backbone (7-90,c12-X, 50%-Sc12) and then independently conjugating 11 different E monomers to it (FIG. 8A). Previous work by the inventors' lab has shown that polymer end-group structure plays an important role in imparting biomaterial-mediated, selective transfection in certain cell types over others, Mishra et al., 2019; Sunshine et al., 2012, and that these effects may be due to changes in NP uptake pathways. Kim et al., 2014. Without wishing to be bound to any one particular theory, it is thought that the presently disclosed dual NP uptake/endosomal escape assay could be useful in further ascertaining how polymer end-groups affect NP performance in different cell types. To test this hypothesis, and to further evaluate the robustness of the presently disclosed high-throughput and high-content bioassay, these polymers were evaluated on 3 cell lines induced to express the Gal8-mRuby construct: B16-F10 murine melanoma cells, RAW 264.7 murine macrophages, and NIH/3T3 murine fibroblasts. These results showed highest mRNA transfection levels in B16-F10 cells, medium transfection in RAW 264.7 macrophages, and lowest transfection levels in NIH/3T3 fibroblasts (FIG. 8A). Endosomal disruption showed positive correlations with mRNA transfection levels in RAW and 3T3 cells, but not B16 cells, with a significant positive correlation when all three cell lines were evaluated together (FIG. 8B). This effect is particularly striking for more difficult-to-transfect cell lines, such as RAW 264.7 and NIH/3T3 cells (Spearman's coefficient of 0.92 and 0.67, respectively), which suggests that mRNA transfection efficacy in difficult-to-transfect cells may largely be attributable to barriers in endosomal escape. Interestingly, the highest NP uptake levels were observed in NIH/3T3 cells, which demonstrated the lowest levels of transfection, and in general mRNA transfection did not show significant correlations with NP uptake (FIG. 8C). Collectively, these results suggest that for PBAEs with the same polymer backbone (and similar hydrophobicity), end-group structure plays an important role in endosomal disruption. These results also indicate that for these PBAEs, endosomal disruption, rather than NP uptake, is acting as a greater bottleneck for effective mRNA delivery. Differing levels of resistance to endosomal disruption among different cell types may also at least partially explain the differential transfection levels among these cells.

### 2.4.4 In Vivo mRNA Delivery: Whole-Body and Organ Level Expression

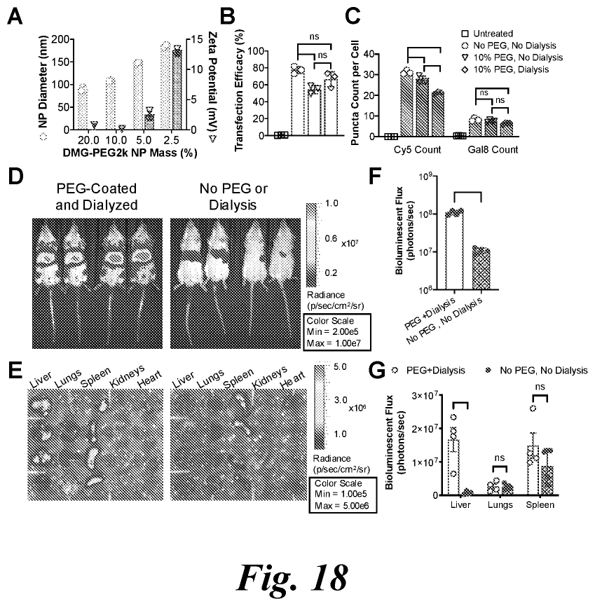

Next, the in vivo mRNA delivery capabilities of PBAE NPs were characterized after intravenous administration of NPs encapsulating mRNA encoding firefly luciferase (fLuc) to mice. For these experiments, NPs were formulated with the PEG-lipid DMG-PEG2k and dialyzed in PBS. Previous incorporation of PEG-lipids into related PBAE NPs has been shown to enhance serum stability and in vivo mRNA expression. Kaczmarek et al., 2018; Eltoukhy et al., 2013. Incorporation of DMG-PEG2k into the PBAE quadpolymers was observed to decrease NP size and neutralize surface charge (FIG. 18). Dialysis and PEG-lipid coating did not significantly change transfection efficacy or endosomal disruption in vitro, though NP uptake was reduced. Upon in vivo administration, PEG-coated and dialyzed NPs enabled significantly higher mRNA expression compared to NPs without PEG coating, and this increased expression was predominately due to increased expression in the liver (FIG. 18F-FIG. 18G).

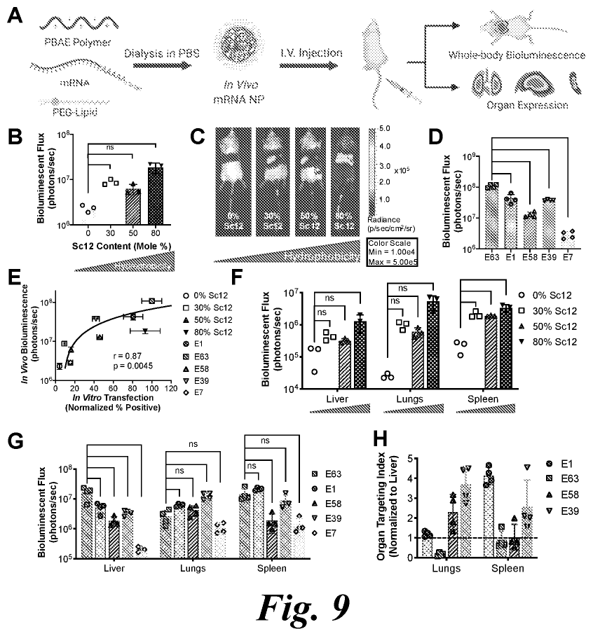

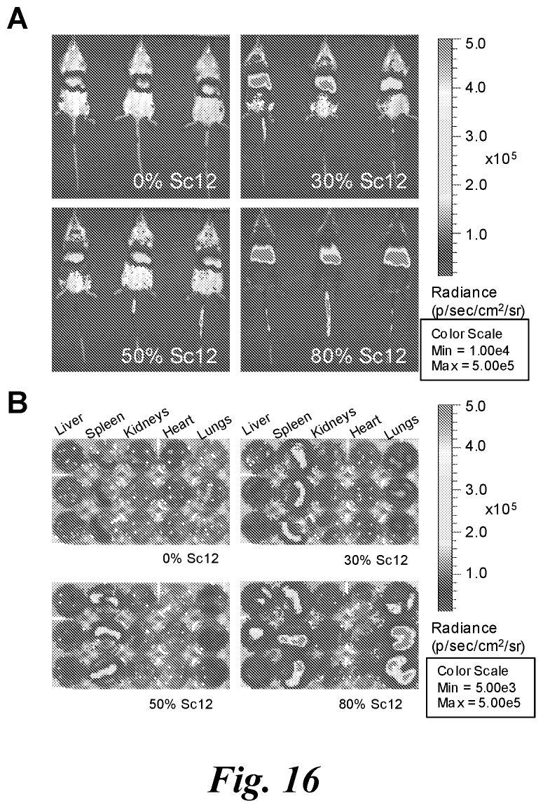

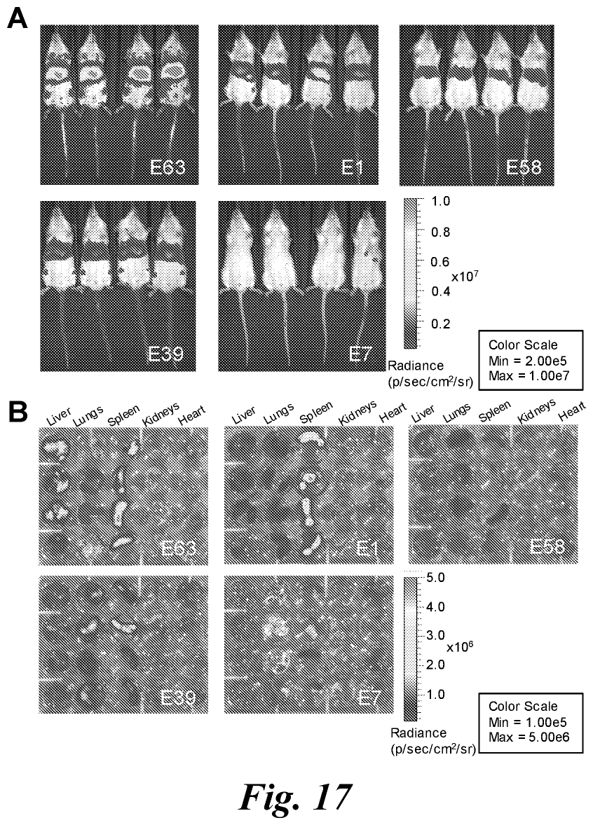

Four polymers with 0-80% Sc12 content in the polymer backbone and five polymers with different polymer end-groups were chosen to assess the effects of polymer backbone and end-group structure, respectively, on in vivo expression. On the whole-body level, increased backbone hydrophobicity generally resulted in increased mRNA expression (FIGS. 9B-9C and FIG. 16) while polymer end-group variation resulted in differential in vivo expression levels (FIG. 9D and FIG. 17). Interestingly, overall in vivo expression correlated positively with in vitro transfection of B16-F10 cells (FIG. 9E), indicating that in vitro screening had predictive capacity for in vivo performance with these nanomaterials. At the level of individual organs, increasing backbone hydrophobicity increased expression in all the organs evaluated (FIG. 9F), while polymer end-group played a major role in targeting NP expression to specific organs (FIG. 9G). Indeed, when expression in the lungs and spleen was normalized to that in the liver, polymer E1 showed preferential expression in the spleen, polymer E63 in the liver, polymer E58 in the lungs, and polymer E39 was almost equally split between the lungs and spleen (FIG. 9H).

### 2.4.5 In Vivo mRNA Delivery: Expression in Different Cell Types

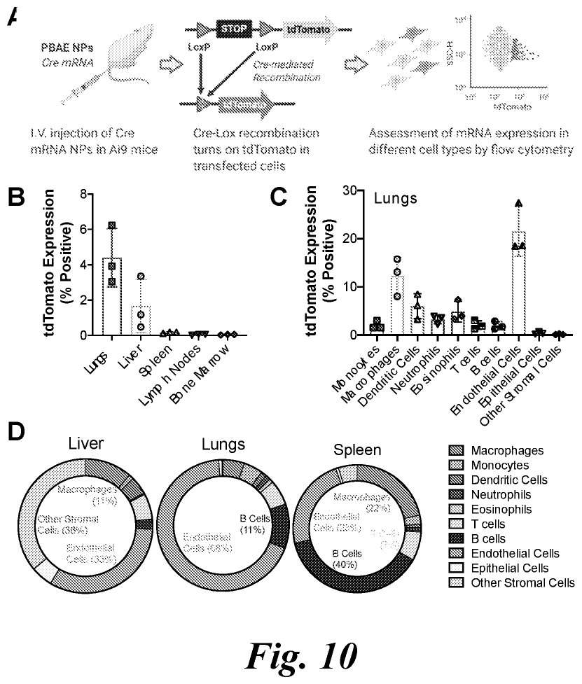

The cell populations that were transfected in each organ were further probed using the Ai9 mouse model, which contains a floxed expression stop cassette upstream of a tdTomato reporter gene. NPs encapsulating Cre mRNA were administered via tail vein injection into Ai9 mice, and transfected cells underwent Cre-Lox recombination, resulting in tdTomato expression that was measured by flow cytometry 3 d post-injection (FIG. 10A). For this study, 7-90,c12-63, 80%-Sc12 NPs were used as they were found to enable high in vivo mRNA expression levels from fLuc mRNA experiments. It was found that 7-90,c12-63, 80%-Sc12 NPs systemically administered transfected nearly 0.2% of the cells in the spleen, 2% of the cells in the liver and 4% of the cells in the lungs, with minimal transfection levels seen in any other organs evaluated (FIG. 10B). Over 20% of endothelial cells in the lungs were transfected following systemic injection, which is consistent with previous reports for related PBAE structures, Kaczmarek et al., 2018, in addition to significant populations of macrophages and dendritic cells in the lungs (FIG. 10C). Endothelial cells also made up a large fraction of the transfected cells in the liver (33%) and spleen (23%) (FIG. 10D).

### 2.5 Discussion

To realize the full therapeutic potential of mRNA therapeutics, a high-throughput, standardized NP screening platform capable of quantitatively evaluating intracellular delivery steps with great predictive capacity for transfection efficacy is needed. In this example, a high-throughput, high-content, imaging-based screening platform designed to simultaneously assess the cellular internalization and endosomal disruption capabilities of nucleic acid delivery NPs was developed, requiring only wide-field, epifluorescence microscopy to enable full assessment of the cytosolic compartment. This bioassay was developed to be implemented in multiwell plates, enabling the evaluation of many intracellular events per cell, in thousands of replicate cells per condition, with up to 96 conditions per plate. Endosomal sequestration has long been identified as a major bottleneck to functional RNA delivery in multiple NP systems, Sahay et al., 2013; Rehman et al., 2013, but quantitative evaluation of endosomal disruption has been limited to low-throughput imaging methods requiring specialized microscopy modalities. Gilleron et al., 2013; Wojnilowicz et al., 2019.

A genetically encoded endosomal disruption sensor based on the natural clustering of Gal8 molecules at damaged endosomal membranes was utilized to detect NP-induced endosomal disruption quantified at the level of intracellular events within single cells. Simultaneously, cellular internalization of NPs could be tracked by delivering nucleic acids labeled with a different fluorophore. Without wishing to be bound to any one particular theory, it was thought that this dual NP uptake and endosomal disruption assay could provide useful information on structure-function relationships when used to screen several NP gene delivery systems.

Two series of PBAE quadpolymers were used to validate this screening platform. PBAEs are cationic, biodegradable polymers that have been shown to be highly effective at in vitro delivery of plasmid DNA, Wilson et al., 2019, siRNA, Karlsson et al., 2019, mRNA, Kaczmarek et al., 2018, and protein cargos. Rui et al., 2019. The highly modular nature of these polymers facilitate combinatorial library synthesis via Michael Addition of small molecule precursors, making it possible to systematically vary polymer backbone or end-group characteristics to directly probe the effects of incremental differential polymer structural changes on downstream nucleic acid delivery efficacy. The PBAE quadpolymer is the majority component of the presently disclosed NP delivery formulations, including systemically administered in vivo formulations, which have 10% PEG-lipid incorporated as a second component, without the presence of other lipids or cholesterol. This approach differs significantly from many previously studied lipid-based NP systems, in which the NP formulation was changed primarily by varying the ratios of incorporated lipids, Sago et al., 2018, or the structure of the ionizable lipid in an NP system consisting of multiple lipid components. Billingsley et al., 2020.

Two polymer series in which polymer backbone hydrophobicity were modulated by varying the content of lipophilic side chain monomers were synthesized to probe the effect of polymer backbone structure on cellular interactions of polymeric NPs. Traditional metrics of predicting NP function, such as polymer nucleic acid binding affinity, endosomal pH buffering potential, NP hydrodynamic diameter, and zeta potential, generally correlated poorly with functional delivery efficacy of multiple nucleic acid cargos, highlighting the need for new metrics for rapid and meaningful NP screening. The dual NP uptake and endosomal disruption assay presented here showed significant correlations with transfection efficacy for all nucleic acid cargos tested. NP uptake correlated positively with transfection (global r=55, p<0.001). Endosomal disruption correlated negatively with transfection for these PBAE NPs (that each had greater endosomal disruption capacity than that achieved by the commercial gene delivery materials) (r=−0.57, p<0.0001). The negative correlation with endosomal disruption is surprising, but may be attributed to the formation of polymer-only NPs that do not contain nucleic acid cargo. Amphiphilic PBAEs like the ones presented in this example have been reported to form polymer-only micellar NPs. Wilson et al., 2017.

Thus, PBAEs that are effective at endosomal disruption, but not efficient at leading to transfection, may be forming large populations of polymer-only NPs empty of nucleic acid cargo. When these polymer-only NPs are internalized by cells, they could enable endosomal disruption, resulting in high Gal8 counts but low transfection. When this dual NP uptake/Gal8 endosomal disruption assay was applied to commercial gene delivery materials such as Lipofectamine 3000, branched and linear PEI, and PLL, endosomal disruption as indicated by Gal8 puncta count was significantly lower for all of these commercial materials than the PBAE NPs, which for the most part also resulted in lower transfection efficacy compared to PBAE NPs. Transfection of these positive control materials correlated positively with endosomal disruption for all cargo types (global r=0.68, p=0.02). Taken together, our data show that a threshold for endosomal disruption, as defined by the amount achieved by the most effective commercial transfection reagent Lipofectamine 3000 (>2 Gal8 puncta per cell in B16-F10 cells), must be reached for gene delivery to efficiently occur. PBAE NPs generally enabled endosomal disruption levels significantly above this threshold in the B16-F10 cells evaluated here and resulted in generally high transfection levels, while commercial materials such as linear PEI and PLL enabled endosomal disruption levels below this threshold and consequently showed negligible transfection levels. The lack of high transfection of PBAE NPs across the board indicates that delivery obstacles further downstream (such as intracellular trafficking or cargo release) may pose significant delivery challenges for some of these materials.

Previous studies have shown that the structure of PBAE polymer end-groups can significantly alter the transfection efficacy of the backbone polymer, as well as impart biomaterial-mediated selectivity in transfection of certain cell types. Kim et al., 2014; Sunshine et al., 2012; Mishra et al., 2019. A polymer series with a common backbone, but with varying end-group structure was synthesized and evaluated for mRNA delivery efficacy on three cell lines. The endosomal disruption levels of these polymers had positive correlations with transfection efficacy, which were stronger in more difficult-to-transfect cell lines as indicated by Spearman's coefficients (r) that are closer to 1; r=0.93 for difficult-to-transfect RAW 264.7 cells, but r=0.47 for easier-to-transfect B16-F10 cells. Differences observed in transfection efficacy were not attributable to polymers' pH buffering capabilities, which varied with backbone structure but were generally unaffected by end-group structure. Even in the 7-90,c12-63×% alkyl side chain polymer series, in which the effective pKa decreased with increasing hydrophobic Sc12 content in the polymer backbone, the correlation between pH buffering and transfection efficacy was poor. This is in contrast to an observation recently reported previously with hyperbranched PBAEs, where increasing polymer branching by incorporation of a triacrylate monomer in the backbone increased both effective pKa and transfection, Wilson et al., 2019, suggesting that different classes of PBAE polymer structures can enable endosomal escape via different mechanisms. In the case of the linear lipophilic PBAE quadpolymers, the endosomal disruption mechanism may rely on the lipophilicity of the polymers causing them to associate with and directly interact with the endosomal membrane, where the charged polymer end-groups may cause transient pore formation that leads to NP leakage out of damaged endosomes, similar to that observed with lipid materials, Rehman et al., 2013, Gilleron et al., 2013, rather than complete endosomal rupture as proposed by the proton sponge hypothesis. Wojnilowicz et al., 2019.

NP uptake of the end-modified linear PBAEs did not correlate significantly with mRNA transfection efficacy (r=0.22, p=0.44), although a significant positive correlation was observed when PBAE NPs carrying each of the three nucleic acid cargos were analyzed globally (global r=0.55, p<0.001). Collectively, these data suggest that endosomal escape is the primary barrier in mRNA delivery to more difficult-to-transfect cells and that the differential gene delivery efficacy mediated by polymer end-groups is largely due to their differential ability to facilitate endosomal disruption.

Finally, these PBAE NPs were validated for in vivo mRNA expression following tail vein injection into mice. NPs formulated by simple mixing of mRNA and polymer in aqueous buffer yielded significantly lower transfection, particularly in the liver, than similar formulations with 10% PEG-lipid dialyzed into the NPs. Using dialyzed PEG-coated formulations, it was observed that in vivo mRNA expression levels correlated strongly with in vitro transfection efficacy in B16-F10 cells, indicating a predictive capacity that is rare in large library screens. Paunovska et al., 2018. Increasing polymer backbone hydrophobicity increased whole-body mRNA expression in general, following trends that were observed in vitro, and which also could be due in part to improved incorporation of PEG-lipid in hydrophobic formulations, which could lead to more stable NPs in the blood. Eltoukhy et al., 2013. Similar to differential transfection of various cell types in vitro, polymer end-group variation also led to tuning of organ tropism in vivo. Unlike most lipid NP formulations which have been demonstrated to predominantly target liver hepatocytes, Akinc et al., 2019; Ramaswamy et al., 2017, the four top performing NP formulations from in vitro mRNA transfection screens in the end-group variation polymer series exhibited different patterns of expression in non-liver organs, with preferential transfection in the lungs and/or spleen. Particularly high expression was seen in the lungs for most formulations, which is consistent with previous reports by Kaczmarek et al., 2018, utilizing similar PBAE lipid-polymer NP formulations for mRNA delivery.

Within each organ, multiple cell types were transfected, including endothelial cells, B cells, and macrophages, all of which have distinct clinical relevance. The lipophilic side chains of the polymers enabled the PEG-lipid DMG-PEG2k to be easily incorporated into NP formulations via dialysis, which increased in vivo expression by an order of magnitude compared to NPs without PEG-lipid coating despite slightly lowering in vitro transfection. Cheng et al. recently reported that incorporation of selective organ targeting (SORT) molecules at defined ratios enabled highly targeted mRNA expression in select organs and that these molecules maintained their organ targeting capabilities across multiple lipid NP platforms. Cheng et al., 2020. This observation suggests intriguing future directions where an innate organ tropism of PBAE NP formulations could perhaps be combined with other technology to enhance selective organ targeting, and other potentially cell-type specific targeting.

In summary, the presently disclosed subject matter provides a high-content high-throughput quantitative imaging assay capable of simultaneously quantifying NP uptake and endosomal disruption. This assay is robust, has higher predictive capacity for in vitro mRNA delivery efficacy compared to conventionally used metrics of polymer or NP properties, and can be performed with approximately 100 nanoparticle formulations in a few hours. Assay validation using PBAE NPs elucidated structure-function relationships through incremental changes in both the polymer backbone and end-groups for these highly modular polymers. Moreover, the presently disclosed subject matter shows that this assay is generally applicable across all major nucleic acid types, several different cell lines, and multiple gene delivery systems. The NP screening platform presented herein can be a useful tool for high-throughput identification of promising candidates for gene delivery and further elucidation of structure/function relationships for the delivery of DNA, siRNA, and mRNA. Lead nanomaterials composed of PBAE quadpolymers demonstrated safe and effective delivery of mRNA in vivo, including organ targeted expression based on polymer structure. PEGylated PBAE NPs enabled significant exogenous mRNA expression differentially to the liver, lung, and spleen. Critically, nanomaterial formulations identified as lead candidates in vitro also performed well for in vivo mRNA delivery following systemic intravenous injection. Such a broadly applicable screening method provides a new metric for nanomaterial characterization, which is important for directly comparing and contextualizing the myriad NP systems that have been reported in the burgeoning field of intracellular gene delivery. With further study, the PBAE-based materials investigated here may be promising for mRNA delivery to promote human health.

### 2.6 Materials and Methods

### 2.6.1 Materials

Bisphenol A glycerolate (1 glycerol/phenol) diacrylate (B7; CAS 4687949), 4-(2-aminoethyl)morpholine (S90; CAS 2038-031), octylamine (Sc8; CAS 111-86-4), 1-decylamine (Sc10; CAS 2016-57-1), oleylamine (Sc18; CAS 112-90-3), 1,3-diaminopropane (E1; CAS 109-76-2), tetraethylenepentamine (E31; CAS 1112-57-2), N,N-diethyldiethylenetriamine (E58; CAS 24426-16-2), tris(2-aminoethyl)amine (E32; CAS 4097-89-6), 2-(3-Aminopropylamino)ethanol (E6; CAS 4461-39-6), 4,7,10-trioxa-1,13-tridecanediamine (E27; CAS 4246-51-9), and 1-(2-aminoethyl)piperazine (E39; CAS 140-31-8) were purchased from Sigma-Aldrich (St. Louis, MO). 1-Dodecylamine (Sc12; CAS 124-22-1) and 1-(3-aminopropyl)-4-methylpiperazine (E7; CAS 4572-031) were purchased from Alfa Aesar (Tewksbury, MA). Tetradecylamine (Sc14; CAS 2016-42-4) and hexadecylamine (Sc16; CAS 143-27-1) were purchased from Acros Organics (Pittsburgh, PA). Diethylenetriamine (E63; CAS 111-40-0) was purchased from EMD Millipore (Burlington, MA). 3,3′-Iminobis(N,N-dimethylpropylamine) (E56; CAS 6711484) was purchased from Santa Cruz Biotechnology (Dallas, TX). 1,4-Bis(3-aminopropyl)piperazine (E65; CAS 7209-38-3) was purchased from MP Biomedicals (Solon, OH).

Plasmid eGFP-N1 (Addgene 2491) was purchased from Elim Biopharmaceuticals (Hayward, CA) and amplified by Aldevron (Fargo, ND). Cy5-labeled plasmid DNA was synthesized following a method reported by Wilson et al., 2017. 5-methoxyuridine-modified CleanCap® eGFP mRNA (L-7201), fLuc mRNA (L-7202), and Cy5-labeled mRNA (L-7702) were purchased from TriLink Biotechnologies (San Diego, CA). Negative control siRNA (1027281) was purchased from Qiagen (Germantown, MD). GFP siRNA targeting the sequence 5′-GCA AGC TGA CCC TGA AGT TC-3′ (P-002048-01) was purchased from Dharmacon (Lafayette, CO). Cy5-labeled siRNA (SIC005) was purchased from Sigma Aldrich (St. Louis, MO). Plasmid DNA encoding a Gal8 fluorescent fusion protein was a generous gift from the lab of Dr. Craig Duvall and cloned into a PiggyBac transposon vector (PB-mRuby3-Gal8, Addgene #150815) for stable integration into mammalian chromosomal DNA.

### 2.6.2 Polymer Synthesis

Polymers were synthesized using previously reported protocols. Wilson et al., 2019. Briefly, diacrylate monomer B7 and side chain monomers (S90 and combinations of ScX monomers) were dissolved at 600 mg/mL in dimethylformamide (DMF) and reacted with stirring for 48 h at 90° C. to allow polymerization via stepwise Michael Addition reactions. Monomers were reacted at an overall vinyl:amine ratio of 2.3 to allow acrylate-terminated polymers to form. Polymers were end-capped by further reaction with primary amine-containing E monomers at room temperature for 2 h [200 mg/mL polymer and 0.3 M E monomer in tetrahydrofuran (THF)] and purified by 2 diethyl ether washes. Diethyl ether was decanted, dried thoroughly under vacuum, and polymers were dissolved in dimethyl sulfoxide (DMSO) at 100 mg/mL and stored at −20° C. with desiccant in single-use aliquots.

### 2.6.3 Polymer Characterization

Polymer molecular weight was characterized using gel permeation chromatography (GPC) against linear polystyrene standards (Waters, Milford, MA). Polymers were dissolved in BHT-stabilized THF and filtered through 0.2-μm PTFE filters prior to GPC measurements. Predicted polymer Log P values were calculated using the online cheminformatics software molinspiration.com.

### 2.6.4 Polymer Buffering Capacity and Determination of Effective pKa

pH titrations were performed using a SevenEasy pH meter (Mettler Toledo, Columbus, OH) as previously described. Wilson et al., 2019. Briefly, 10 mg polymer was dissolved in 10 mL of 100 mM NaCl acidified with HCl and titrated from pH 3.0 to pH 11.0 via stepwise addition of 100 mM NaOH. To calculate the effective pKa of the polymer in the physiologically relevant pH range (pH 5-8), normalized buffering capacity was calculated from titration data as Δ(—OH)/Δ(pH) for each titration point. Effective pKa was defined as the pH point corresponding to the maximum normalized buffering capacity.

### 2.6.5 Nucleic Acid Binding Assays

Ribogreen nucleic acid binding dye (Invitrogen, Carlsbad, CA) was mixed with nucleic acids in 25 mM magnesium acetate buffer (MgAc₂, pH 5.0) at a final nucleic acid concentration of 5 μg/mL (siRNA), 2.5 μg/mL (mRNA), or 1 μg/mL (pDNA) and a final 1:2000 RiboGreen dilution. Polymers were dissolved and serially diluted to a range of concentrations in MgAc₂, and 25 μL polymer solution was mixed with 75 μL nucleic acid/RiboGreen solution per well in 96-well black bottom assay plates. The solutions were incubated at 37° C. for 20 minutes before fluorescence readings were taken on a Biotek Synergy 2 fluorescence multiplate reader (BioTek, Winooski, VT). To characterize nucleic acid binding affinity, the polymer IC₅₀ of binding (polymer concentration at which 50% of RiboGreen fluorescence is quenched by RiboGreen displacement from polymer binding to nucleic acids) was calculated by plotting % fluorescence quenching as a function of polymer concentration and fitting a sigmoidal curve to the data. Polymer IC₅₀ of binding varies inversely with binding affinity; lower IC₅₀ values indicate higher binding affinity.

### 2.6.6 NP Formulation and Characterization

For in vitro studies, NPs were formulated in 25 mM magnesium acetate buffer (MgAc₂, pH 5) and added directly to cells without the addition of PEG lipids or dialysis. Polymers and nucleic acids (plasmid DNA, mRNA, or siRNA) were dissolved separately in 25 mM MgAc₂ at concentrations of 0.83 ng/μL for nucleic acids and 50 ng/μL for polymers, and mixed together via pipetting at a 1:1 volume ratio. NPs were allowed to self-assemble for 10 minutes at room temperature; the polymer-to-nucleic acid ratio was 60 by weight (60 w/w) for all experiments.

NP hydrodynamic diameter was measured via dynamic light scattering (DLS) using a Malvern Zetasizer Pro with universal dip cell (Malvern Panalytical, Malvern, United Kingdom). Samples were prepared in 25 mM MgAc₂ and diluted 1:6 in 150 mM PBS to determine NP characteristics in neutral, isotonic buffer. Zeta potential was measured by electrophoretic light scattering on the same instrument. Transmission electron microscopy (TEM) images were captured using a Philips CM120 transmission electron microscope (Philips Research, Cambridge, MA). 30 μL NP samples were allowed to coat 400-square mesh carbon coated TEM grids for 20 minutes. Grids were then rinsed with ultrapure water and allowed to fully dry before imaging.

### 2.6.7 Cell Culture and Cell Line Preparation

B16-F10 murine melanoma and RAW 264.7 murine macrophage cells were cultured in Dulbecco's Modified Eagle Medium (DMEM; ThermoFisher, Waltham, MA) supplemented with 10% FBS and 1% penicillin/streptomycin. GFPd2+ B16-F10 cells used in siRNA knockdown experiments were established previously, Rui et al., 2019, and cultured using the same medium. NIH/3T3 murine fibroblasts were cultured in DMEM supplemented with 10% bovine calf serum and 1% penicillin/streptomycin. Cells were induced to constitutively express the Gal8-mRuby fusion fluorescent protein construct using the PiggyBac transposon/transposase system. The PiggyBac transposon plasmid carrying the Gal8-mRuby gene was created using restriction enzyme cloning and is available on Addgene (plasmid #150815). The transposase expression plasmid (PB200A-1) was purchased from System Biosciences (Palo Alto, CA). The transposon plasmid was co-transfected with the PiggyBac transposase plasmid using PBAE NPs as described below. mRuby+ cells were isolated using at least two rounds of fluorescence assisted cell sorting using a Sony SH800 Cell Sorter (Sony Biotechnology, San Jose, CA) to generate stably expressing cell lines.

### 2.6.8 Transfection

Cells were plated at 10,000 cells per well in 100 μL complete medium in CytoOne 96 well plates (USA Scientific, Ocala, FL) and allowed to adhere overnight. NPs were formulated following the in vitro transfection formulation described above; 20 μL NP solution was added to 100 μL fresh complete medium, and 120 μL per well of the NP medium mixture was used to replace the culture medium. For all in vitro transfections, NPs were formulated at 60 w/w delivering 50 ng nucleic acids per well. For cellular uptake experiments, 20% of the total nucleic acid drugs were replaced with Cy5-labeled nucleic acids prior to mixing with polymers. NPs were incubated with cells at 37° C. for the appropriate duration, depending on assay conditions (6 h for dual uptake/Gal8 assay, 24 h for mRNA and siRNA transfections, and 48 h for DNA transfections).

For transfections using commercially available reagents, Lipofectamine™ 3000 (ThermoFisher) was used as instructed by the manufacturer. 25 kD branched polyethylenimine (BPEI), 2.5 kD linear polyethylenimine (LPEI), and 15 kD poly-L-lysine (PLL) were used at the highest concentrations that did not cause significant cytotoxicity (15 w/w for BPEI, 60 w/w for LPEI, and 30 w/w for PLL). PEI NPs were formulated in 150 mM NaCl solution, and PLL NPs were formulated in 10 M HEPES buffer (pH 7); all formulations delivered 50 ng nucleic acids to match the dose delivered by PBAE NPs.

Transfection efficacy was evaluated via flow cytometry using a BD Accuri C6 flow cytometer (BD Biosciences, East Rutherford, NJ). For plasmid DNA and mRNA transfections, the expression of a GFP reporter gene was quantified by normalizing the geometric mean fluorescence intensity of each NP treatment to that of the formulation achieving maximum expression. Cells previously engineered to constitutively express GFP, Rui et al, ACS Applied Materials & Interfaces, 2019, were used for siRNA knockdown transfections and the percentage of cells positively expressing GFP when gated against untreated cells in wells treated with siRNA targeting GFP was normalized against that of wells treated with non-coding control siRNA.

### 2.6.9 Dual NP Uptake and Gal8 Endosomal Disruption Assay

NPs of matching formulation as those used for transfection experiments were used to deliver nucleic acids cargo containing 20% Cy5-labeled nucleic acids to enable visualization of NP uptake. NPs were incubated with Gal8-mRuby+ cells for 6 h (assay time point optimized in FIG. 12), at which point NPs and cell culture medium were removed, cells were washed with PBS, and fixed with 10% formalin for minutes at room temperature. The formalin was then removed, cells washed with PBS, and Hoechst nuclear stain (1:5000 in PBS) was applied for 10 minutes. NP uptake and Gal8-mRuby endosomal escape were then quantified by high-content imaging analysis of Cy5 and mRuby puncta per cell, respectively, using a CellInsight CX7 LZR high-content imager (ThermoFisher) with HCS Studio analysis software.

### 2.6.10 NP Formulation for In Vivo Studies

NPs for in vivo mRNA delivery were formulated at 30 w/w. mRNA was dissolved in MgAc₂, while polymer and the PEG-lipid 1,2-dimyristoyl-rac-glycero-3-methoxypolyethylene glycol-2000 (DMG-PEG2k, 10% by mass) were dissolved in 100% ethanol. The mRNA and polymer-PEG lipid solutions were mixed via pipetting at 1:1 volume ratio, and NPs were allowed to self-assemble at room temperature for minutes. NPs were then dialyzed against cold PBS at 4° C. for 75 minutes using Spectra/Por Float-A-Lyzer G2 dialysis devices (Repligen, Waltham, MA) with 50 kD molecular weight cut-off. NP volume post-dialysis was adjusted with PBS for final mRNA concentration of 0.1 mg/mL. NPs were administered to animals via 100 μL tail vein injections for a final dose of 10 lag mRNA per animal.

To investigate the effects of PEGylation and dialysis on in vivo mRNA expression, NPs with no PEG lipid and no dialysis were formulated in 25 mM MgAc₂ at the same final mRNA concentration and w/w ratio as above. 500 mg/mL sucrose solution was used to bring the mixture to isotonicity.

### 2.6.11 fLuc mRNA In Vivo Bioluminescence

NPs encapsulating fLuc mRNA were formulated as described above and administered to 6-7 week old male BALB/c mice via lateral tail vein injection. Whole-body bioluminescence was assessed 24 h post-injection. D-luciferin potassium salt solution (25 mg/mL in PBS; Cayman Chemical Company, Ann Arbor, MI) was administered to mice via 150 μL intraperitoneal injection, and mice were imaged using an IVIS Spectrum Imager (Perkin Elmer, Waltham, MA) 10 minutes later. The same animals were euthanized immediately after whole-body imaging via cervical dislocation, and select organs were extracted, submerged in 250 μg/mL D-luciferin solution, and imaged with IVIS.

### 2.6.12 Cre mRNA Delivery to Ai9 Mice

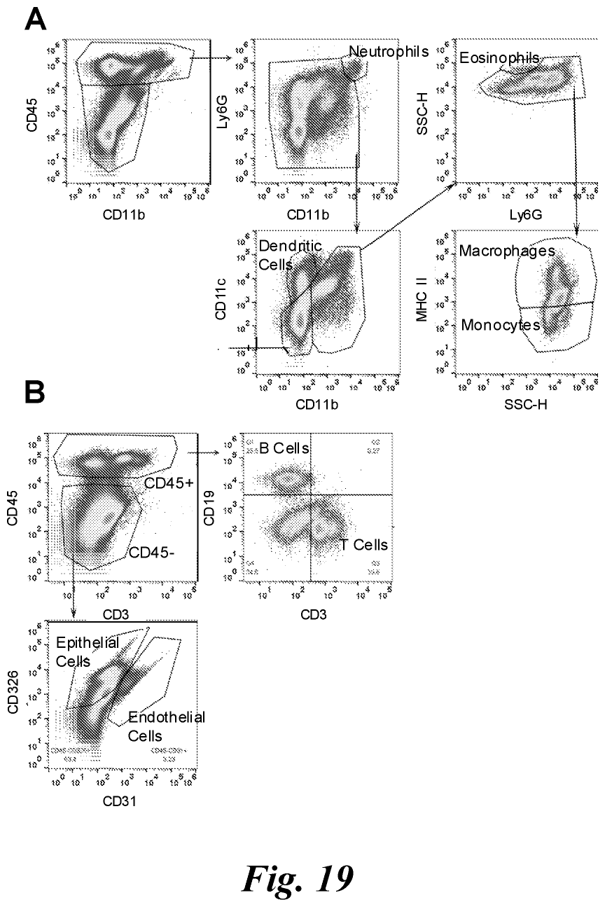

NPs encapsulating Cre mRNA were formulated with DMG-PEG2k and dialyzed in PBS as described above. NPs were administered to 6-week old male Ai9 mice via tail vein injection, and tdTomato expression following Cre-Lox recombination was allowed to accumulate for 3 days, at which point animals were euthanized via cervical dislocation. Select organs were extracted and dissociated by a 1 hr incubation in 2 mg/mL collagenase at 37° C. followed by mechanical pressing through a 70-μm cell strainer. Cells were pelleted by centrifugation, the supernatant was removed, and red blood cells in the cell pellet were lysed by incubating in ACK lysing buffer (Quality Biological, Gaithersburg, MD) for 1 min at room temperature. Cells were diluted in PBS, passed through a 100-nm cell strainer, pelleted by centrifugation, and resuspended in FACS buffer (2% FBS in PBS with 0.02% sodium azide). Surface staining of cells with fluorescent antibodies was then performed using the antibodies and dilutions listed in Table 1 in FACS buffer for 30 min at 4° C., at which time cells were washed twice and resuspended in FACS buffer for further analysis. FACS experiments were performed using an Attune NxT flow cytometer (ThermoFisher) and analyzed using FlowJo software (FlowJo, Ashland, OR). Gating strategies to identify cell populations are provided in FIG. 19.

TABLE 1
Antibody information for Ai9 flow cytometry experiments.

| Antigen | Color | Supplier | Clone | Dilution | Catalog No. | Lot No |
|---|---|---|---|---|---|---|
| CD45 | Brilliant Violet 421 | Biolegend | 30-F11 | 1:100 | 103134 | B287242 |
| CD11b | Alexa Fluor 488 | Biolegend | M1/70 | 1:100 | 101217 | B254608 |
| CD11c | Allophycocyanin (APC) | Biolegend | N418 | 1:100 | 117310 | B278343 |
| I-A/I-E | Alexa Fluor 700 | Biolegend | M5/114.14.2 | 1:100 | 107622 | B264454 |
| Ly6G | APC/Cyanine7 (Cy7) | Biolegend | 1A8 | 1:100 | 127624 | B264760 |
| CD3 | Alexa Fluor 488 | Biolegend | 17A2 | 1:80 | 100210 | B284975 |
| CD19 | APC | Biolegend | 6D5 | 1:100 | 115512 | B284257 |
| CD31/PECAM | Alexa Fluor 700 | Biolegend | 390 | 1:100 | 102443 | B303280 |
| CD326/EpCAM | APC/Cy7 | Biolegend | G8.8 | 1:80 | 118218 | B266989 |

### 2.6.13 Data Analysis and Statistics

Curve plotting and statistical analysis were performed using Prism 8 (Graphpad, La Jolla, CA). Data are shown as mean±SD for groups of three or more replicates or as individual values with the mean indicated. Unless otherwise stated, absence of statistical significance markings where a test was stated to have been performed signify no statistical significance. The statistical tests used for each figure are indicated in the figure captions. Statistical significance is denoted as follows: *p<0.05; **p<0.01, ***p<0.001, ****p<0.0001. ns=not significant.

### 2.6.14 Graphical Illustrations

Graphical illustrations were created using BioRender (https://biorender.com/).

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Karikó, K.; Muramatsu, H.; Welsh, F. A.; Ludwig, J.; Kato, H.; Akira, S.; Weissman, D., Incorporation of pseudouridine into mRNA yields superior nonimmunogenic vector with increased translational capacity and biological stability. Molecular therapy 2008, 16 (11), 1833-1840.
Thess, A.; Grund, S.; Mui, B. L.; Hope, M. J.; Baumhof, P.; Fotin-Mleczek, M.; Schlake, T., Sequence-engineered mRNA without chemical nucleoside modifications enables an effective protein therapy in large animals. Molecular Therapy 2015, 23 (9), 1456-1464.
Corbett et al., SARS-CoV-2 mRNA vaccine design enabled by prototype pathogen preparedness. Nature 2020, 586, 567-571.
Pardi, N.; Hogan, M. J.; Porter, F. W.; Weissman, D., mRNA vaccines—a new era in vaccinology. Nature Reviews Drug Discovery 2018, 17 (4), 261-279.
Mandal, P. K.; Ferreira, L. M. R.; Collins, R.; Meissner, T. B.; Boutwell, C. L.; Friesen, M.; Vrbanac, V.; Garrison, B. S.; Stortchevoi, A.; Bryder, D., Efficient ablation of genes in human hematopoietic stem and effector cells using CRISPR/Cas9. Cell stem cell 2014, 15 (5), 643-652.
Monroe, K. M.; Yang, Z.; Johnson, J. R.; Geng, X.; Doitsh, G.; Krogan, N. J.; Greene, W. C., IFI16 DNA sensor is required for death of lymphoid CD4 T cells abortively infected with HIV. Science 2014, 343 (6169), 428-432.
Sabnis, S.; Kumarasinghe, E. S.; Salerno, T.; Mihai, C.; Ketova, T.; Senn, J. J.; Lynn, A.; Bulychev, A.; McFadyen, I.; Chan, J., A novel amino lipid series for mRNA delivery: improved endosomal escape and sustained pharmacology and safety in non-human primates. Molecular Therapy 2018, 26 (6), 1509-1519.
Patel, A. K.; Kaczmarek, J. C.; Bose, S.; Kauffman, K. J.; Mir, F.; Heartlein, M. W.; DeRosa, F.; Langer, R.; Anderson, D G, Inhaled Nanoformulated mRNA Polyplexes for Protein Production in Lung Epithelium. Advanced Materials 2019, 31 (8), 1805116.
Cheng, Q.; Wei, T.; Jia, Y.; Farbiak, L.; Zhou, K.; Zhang, S.; Wei, Y.; Zhu, H.; Siegwart, D. J., Dendrimer-Based Lipid Nanoparticles Deliver Therapeutic FAH mRNA to Normalize Liver Function and Extend Survival in a Mouse Model of Hepatorenal Tyrosinemia Type I. Advanced Materials 2018, 30 (52), 1805308.
Cao, J.; An, D.; Galduroz, M.; Zhuo, J.; Liang, S.; Eybye, M.; Frassetto, A.; Kuroda, E.; Funahashi, A.; Santana, J.; Mihai, C.; Benenato, K. E.; Kumarasinghe, E. S.; Sabnis, S.; Salerno, T.; Coughlan, K.; Miracco, E. J.; Levy, B.; Besin, G.; Schultz, J.; Lukacs, C.; Guey, L.; Finn, P.; Furukawa, T.; Giangrande, P. H.; Saheki, T.; Martini, P. G. V., mRNA Therapy Improves Metabolic and Behavioral Abnormalities in a Murine Model of Citrin Deficiency. Molecular Therapy 2019, 27 (7), 1242-1251.
Billingsley, M. M.; Singh, N.; Ravikumar, P.; Zhang, R.; June, C. H.; Mitchell, M. J., Ionizable Lipid Nanoparticle-Mediated mRNA Delivery for Human CAR T Cell Engineering. Nano Letters 2020, 20 (3), 1578-1589.
Miao, L.; Li, L.; Huang, Y.; Delcassian, D.; Chahal, J; Han, J.; Shi, Y.; Sadtler, K.; Gao, W.; Lin, J.; Doloff, J. C.; Langer, R.; Anderson, D. G., Delivery of mRNA vaccines with heterocyclic lipids increases anti-tumor efficacy by STING-mediated immune cell activation. Nature Biotechnology 2019, 37 (10), 1174-1185.
Liu, J.; Chang, J.; Jiang, Y.; Meng, X.; Sun, T.; Mao, L.; Xu, Q.; Wang, M., Fast and efficient CRISPR/Cas9 genome editing in vivo enabled by bioreducible lipid and messenger RNA nanoparticles. Advanced Materials 2019, 31 (33), 1902575.
Miller, J. B.; Zhang, S.; Kos, P.; Xiong, H.; Zhou, K.; Perelman, S. S.; Zhu, H.; Siegwart, D. J., Non-viral CRISPR/Cas gene editing in vitro and in vivo enabled by synthetic nanoparticle co-delivery of Cas9 mRNA and sgRNA. Angewandte Chemie International Edition 2017, 56 (4), 1059-1063.
Rui, Y.; Wilson, D. R.; Green, J. J., Non-Viral Delivery To Enable Genome Editing. Trends in Biotechnology 2019, 37 (3), 281-293.
Gilleron, J.; Querbes, W.; Zeigerer, A.; Borodovsky, A.; Marsico, G.; Schubert, U.; Manygoats, K.; Seifert, S.; Andree, C.; Stoter, M.; Epstein-Barash, H.; Zhang, L.; Koteliansky, V.; Fitzgerald, K.; Fava, E.; Bickle, M.; Kalaidzidis, Y.; Akinc, A.; Maier, M.; Zerial, M., Image-based analysis of lipid nanoparticle—mediated siRNA delivery, intracellular trafficking and endosomal escape. Nature Biotechnology 2013, 31 (7), 638-646.
Tamura, A.; Oishi, M.; Nagasaki, Y., Enhanced Cytoplasmic Delivery of siRNA Using a Stabilized Polyion Complex Based on PEGylated Nanogels with a Cross-Linked Poly amine Structure. Biomacromolecules 2009, 10 (7), 1818-1827.
Akita, H.; Kogure, K.; Moriguchi, R.; Nakamura, Y.; Higashi, T.; Nakamura, T.; Serada, S.; Fujimoto, M.; Naka, T.; Futaki, S.; Harashima, H., Nanoparticles for ex vivo siRNA delivery to dendritic cells for cancer vaccines: Programmed endosomal escape and dissociation. Journal of Controlled Release 2010, 143 (3), 311-317.
Kilchrist, K. V.; Evans, B. C.; Brophy, C. M.; Duvall, C. L., Mechanism of Enhanced Cellular Uptake and Cytosolic Retention of MK2 Inhibitory Peptide Nano-polyplexes. Cell Mol Bioeng 2016, 9 (3), 368-381.
Wittrup, A.; Ai, A.; Liu, X.; Hamar, P.; Trifonova, R.; Charisse, K.; Manoharan, M.; Kirchhausen, T.; Lieberman, J., Visualizing lipid-formulated siRNA release from endosomes and target gene knockdown. Nature biotechnology 2015, 33 (8), 870-876.
Wojnilowicz, M.; Glab, A.; Bertucci, A.; Caruso, F.; Cavalieri, F., Super-resolution Imaging of Proton Sponge-Triggered Rupture of Endosomes and Cytosolic Release of Small Interfering RNA. ACS Nano 2019, 13 (1), 187-202.
Hadari, Y. R.; Paz, K.; Dekel, R.; Mestrovic, T.; Accili, D.; Zick, Y., Galectin-8: A NEW RAT LECTIN, RELATED TO GALECTIN-4. Journal of Biological Chemistry 1995, 270 (7), 3447-3453.
Thurston, T. L. M.; Wandel, M. P.; Von Muhlinen, N.; Foeglein, Á.; Randow, F., Galectin 8 targets damaged vesicles for autophagy to defend cells against bacterial invasion. Nature 2012, 482 (7385), 414-418.
Kilchrist, K. V.; Dimobi, S. C.; Jackson, M. A.; Evans, B. C.; Werfel, T. A.; Dailing, E. A.; Bedingfield, S. K.; Kelly, I. B.; Duvall, C. L., Gal8 Visualization of Endosome Disruption Predicts Carrier-Mediated Biologic Drug Intracellular Bioavailability. ACS Nano 2019, 13 (2), 1136-1152.
Wittrup, A.; Ai, A.; Liu, X.; Hamar, P.; Trifonova, R.; Charisse, K.; Manoharan, M.; Kirchhausen, T.; Lieberman, J., Visualizing lipid-formulated siRNA release from endosomes and target gene knockdown. Nature Biotechnology 2015, 33 (November 2014), 1-9.
Kaczmarek, J. C.; Kauffman, K. J.; Fenton, O. S.; Sadtler, K.; Patel, A. K.; Heartlein, M. W.; DeRosa, F.; Anderson, D. G., Optimization of a Degradable Polymer—Lipid Nanoparticle for Potent Systemic Delivery of mRNA to the Lung Endothelium and Immune Cells. Nano Letters 2018, 18 (10), 6449-6454.
Eltoukhy, A. A.; Chen, D.; Alabi, C. A.; Langer, R.; Anderson, D. G., Degradable terpolymers with alkyl side chains demonstrate enhanced gene delivery potency and nanoparticle stability. Advanced Materials 2013, 25 (10), 1487-1493.
Sahay, G.; Querbes, W.; Alabi, C.; Eltoukhy, A.; Sarkar, S.; Zurenko, C.; Karagiannis, E.; Love, K.; Chen, D.; Zoncu, R.; Buganim, Y.; Schroeder, A.; Langer, R.; Anderson, D. G., Efficiency of siRNA delivery by lipid nanoparticles is limited by endocytic recycling. Nature Biotechnology 2013, 31 (7), 653-658.
Rehman, Z. u.; Hoekstra, D.; Zuhorn, I. S., Mechanism of Polyplex- and Lipoplex-Mediated Delivery of Nucleic Acids: Real-Time Visualization of Transient Membrane Destabilization without Endosomal Lysis. ACS Nano 2013, 7 (5), 3767-3777.
Wilson, D. R.; Rui, Y.; Siddiq, K.; Routkevitch, D.; Green, J. J., Differentially Branched Ester Amine Quadpolymers with Amphiphilic and pH-Sensitive Properties for Efficient Plasmid DNA Delivery. Molecular Pharmaceutics 2019, 16 (2), 655-668.
Karlsson, J.; Rui, Y.; Kozielski, K. L.; Placone, A. L.; Choi, O.; Tzeng, S. Y.; Kim, J.; Keyes, J. J.; Bogorad, M. I.; Gabrielson, K.; Guerrero-Cazares, H.; Quiñones-Hinojosa, A.; Searson, P. C.; Green, J. J., Engineered nanoparticles for systemic siRNA delivery to malignant brain tumours. Nanoscale 2019, 11 (42), 20045-20057.
Rui, Y.; Wilson, D. R.; Choi, J.; Varanasi, M.; Sanders, K.; Karlsson, J.; Lim, M.; Green, J. J., Carboxylated branched poly(β-amino ester) nanoparticles enable robust cytosolic protein delivery and CRISPR-Cas9 gene editing. Science Advances 2019, 5 (12), eaay3255.
Sago, C. D.; Lokugamage, M. P.; Paunovska, K.; Vanover, D. A.; Monaco, C. M.; Shah, N. N.; Gamboa Castro, M.; Anderson, S. E.; Rudoltz, T. G.; Lando, G. N.; Munnilal Tiwari, P.; Kirschman, J. L.; Willett, N.; Jang, Y. C.; Santangelo, P. J.; Bryksin, A. V.; Dahlman, J. E., High-throughput in vivo screen of functional mRNA delivery identifies nanoparticles for endothelial cell gene editing. Proceedings of the National Academy of Sciences 2018, 115 (42), E9944.
Wilson, D. R.; Mosenia, A.; Suprenant, M. P.; Upadhya, R.; Routkevitch, D.; Meyer, R. A.; Quinones-Hinojosa, A.; Green, J. J., Continuous microfluidic assembly of biodegradable poly(beta-amino ester)/DNA nanoparticles for enhanced gene delivery. Journal of Biomedical Materials Research Part A 2017, 105 (6), 1813-1825.
Kim, J.; Sunshine, J. C.; Green, J. J., Differential polymer structure tunes mechanism of cellular uptake and transfection routes of poly((3-amino ester) polyplexes in human breast cancer cells. Bioconjugate chemistry 2014, 25 (1), 43-51.
Sunshine, J. C.; Peng, D. Y.; Green, J. J., Uptake and Transfection with Polymeric Nanoparticles Are Dependent on Polymer End-Group Structure, but Largely Independent of Nanoparticle Physical and Chemical Properties. Molecular Pharmaceutics 2012, 9 (11), 3375-3383.
Mishra, B.; Wilson, D. R.; Sripathi, S. R.; Suprenant, M. P.; Rui, Y.; Wahlin, K. J.; Berlinicke, C. A.; Green, J. J.; Zack, D. J., A Combinatorial Library of Biodegradable Polyesters Enables Non-viral Gene Delivery to Post-Mitotic Human Stem Cell-Derived Polarized RPE Monolayers. Regenerative Engineering and Translational Medicine 2019.
Paunovska, K.; Sago, C. D.; Monaco, C. M.; Hudson, W. H.; Castro, M. G.; Rudoltz, T. G.; Kalathoor, S.; Vanover, D. A.; Santangelo, P. J.; Ahmed, R.; Bryksin, A. V.; Dahlman, J E, A Direct Comparison of in Vitro and in Vivo Nucleic Acid Delivery Mediated by Hundreds of Nanoparticles Reveals a Weak Correlation. Nano Letters 2018, 18 (3), 2148-2157.
Akinc, A.; Maier, M. A.; Manoharan, M.; Fitzgerald, K.; Jayaraman, M.; Barros, S.; Ansell, S.; Du, X.; Hope, M. J.; Madden, T. D.; Mui, B. L.; Semple, S. C.; Tam, Y. K.; Ciufolini, M.; Witzigmann, D; Kulkarni, J. A.; van der Meel, R.; Cullis, P. R., The Onpattro story and the clinical translation of nanomedicines containing nucleic acid-based drugs. Nature Nanotechnology 2019, 14 (12), 1084-1087.
Ramaswamy, S.; Tonnu, N.; Tachikawa, K.; Limphong, P.; Vega, J. B.; Karmali, P. P.; Chivukula, P.; Verma, I. M., Systemic delivery of factor IX messenger RNA for protein replacement therapy. Proceedings of the National Academy of Sciences 2017, 114 (10), E1941.
Cheng, Q.; Wei, T.; Farbiak, L.; Johnson, L. T.; Dilliard, S. A.; Siegwart, D. J., Selective organ targeting (SORT) nanoparticles for tissue-specific mRNA delivery and CRISPR-Cas gene editing. Nature Nanotechnology 2020, 15 (4), 313-320.
Wilson, D. R.; Routkevitch, D.; Rui, Y.; Mosenia, A.; Wahlin, K. J.; Quinones-Hinojosa, A.; Zack, D. J.; Green, J. J., A Triple-Fluorophore-Labeled Nucleic Acid pH Nanosensor to Investigate Non-viral Gene Delivery. Molecular Therapy 2017, 25 (7), 1697-1709.
Rui, Y.; Wilson, D. R.; Sanders, K.; Green, J. J., Reducible Branched Ester-Amine Quadpolymers (rBEAQs) Codelivering Plasmid DNA and RNA Oligonucleotides Enable CRISPR/Cas9 Genome Editing. ACS Applied Materials and Interfaces 2019, 11(11), 10472-10480.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A composition comprising a compound of formula (I): 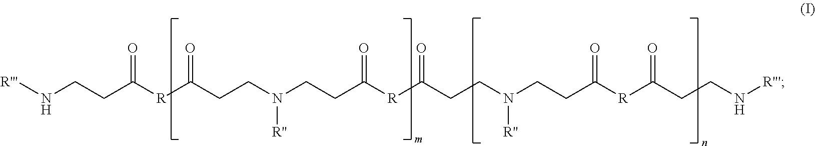
wherein:
m and n are each integers from 1 to 10,000;
R is derived from a linear diacrylate;
R′ is derived from a hydrophobic amine;
R″ is derived from a hydrophilic amine; and
R′″ is an end-capping group.

2. The composition of claim 1, wherein the linear diacrylate comprises:

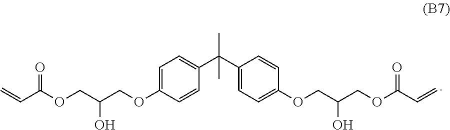 [SMILES: C=CC(=O)OCC(O)COc1ccc(C(C)(C)c2ccc(OCC(O)COC(=O)C=C)cc2)cc1]

3. The composition of claim 1, wherein the hydrophobic amine comprises: 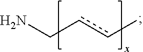
wherein x is an integer selected from 0, 1, 2, 3, 4, 5, 6, 7, 8, 9, 10, 11, 12, 13, 14, 15, 16, 17, 18, 19, and 20; and
wherein 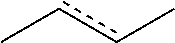
can be a single or double bond in one or more x repeating units.

4. The composition of claim 3, wherein the hydrophobic amine is selected from the group consisting of:

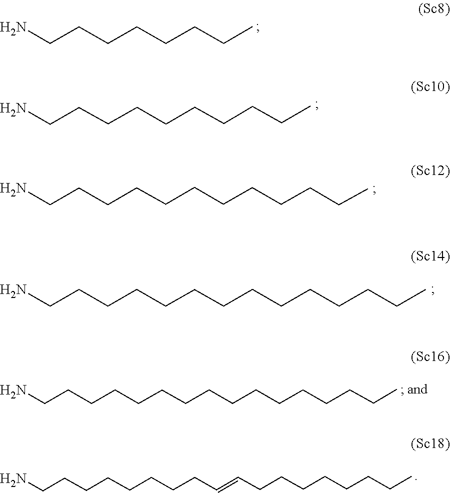

5. The composition of claim 1, wherein the hydrophilic amine comprises:

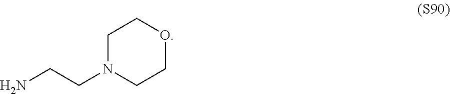

6. The composition of claim 1, wherein the end-capping group is selected from the group consisting of:

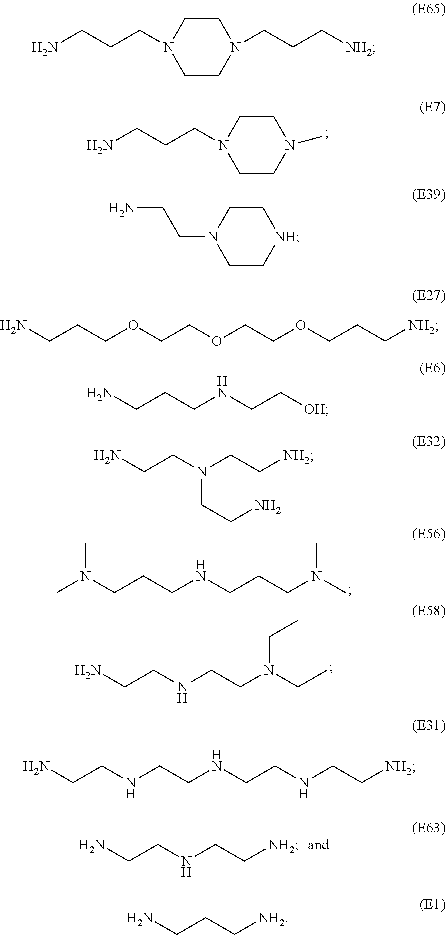

7. The composition of claim 6, wherein the end-capping group is:

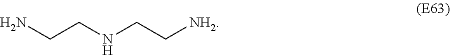

8. The composition of claim 1, wherein the linear diacrylate is B7 and the hydrophobic amine is a blend of S90 and Sc12 and the end-capping group is selected from the group consisting of:

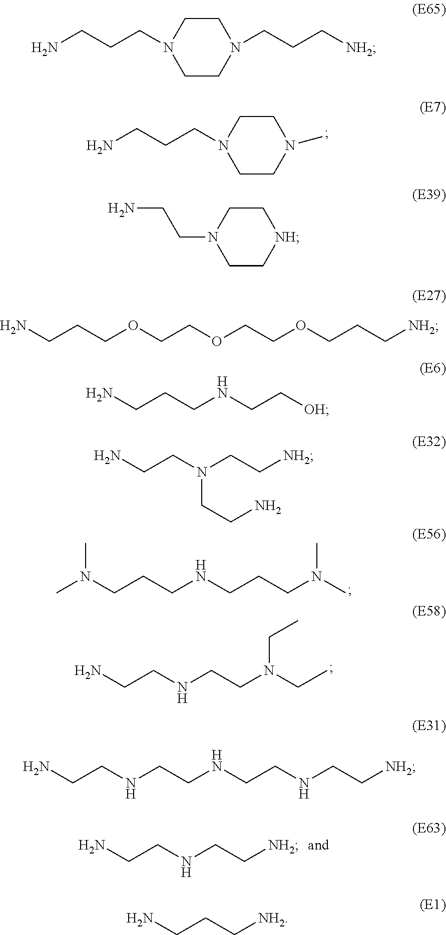

9. The composition of claim 1, wherein the linear diacrylate is B7, the end-capping group is E63, the hydrophilic amine is S90, and the hydrophobic amine is selected from the group consisting of S8, S10, S12, S14, S16, and S18.

10. The composition of claim 9, wherein at least one of S8, S10, S12, S14, S16, and S18 is present at a percentage ranging from about 15% to 80% relative to a percentage of S90.

11. The composition of any one of claims 1-10, further comprising one or more nucleic acids.

12. The composition of claim 11, wherein the one or more nucleic acids is selected from the group consisting of mRNA, DNA, an oligonucleotide, a cyclic dinucleotide, other small nucleic acids, and combinations thereof.

13. The composition of any one of claims 1-12, further comprising PEG-lipid.

14. The composition of claim 13, comprising from about 0% to about 15% PEG-lipid.

15. The composition of claim 14, wherein the end-capping group is selected from the group consisting of E63, E1, E58, E39, and E7.

16. A formulation comprising the composition of any one of claims 1-15, wherein the formulation is one or more of frozen, lyophilized, or combined with one or more excipients to extend stability.

17. A nanoparticle comprising the composition of any one of claims 1-15.

18. The nanoparticle of claim 17, wherein the nanoparticle is targeted for a tissue.

19. The nanoparticle of claim 17, wherein the nanoparticle comprises greater than about 50% of a dry particle mass.

20. A method for systemic delivery of mRNA to a tissue, the method comprising administering a composition of any one of claims 1-15, a formulation of claim 16, or a nanoparticle of any one of claims 17-19 to the tissue.

21. The method of claim 20, wherein the tissue comprises tissue from an organ selected from the group consisting of lung, liver, kidney, heart, and spleen.

22. A method for systemic deliver of mRNA to one or more immune cells, the method comprising administering a composition of any one of claims 1-15, a formulation of claim 16, or a nanoparticle of any one of claims 17-19 to the one or more immune cells.

23. A method for treating a disease, condition, or disorder, the method comprising administering to a subject in need of treatment thereof a composition of any one of claims 1-15, a formulation of claim 16, or a nanoparticle of any one of claims 17-19.

24. The method of claim 23, wherein the composition or nanoparticle comprises one or more of mRNA, plasmid DNA, an oligonucleotide, a cyclic dinucleotide, other small nucleic acids, and combinations thereof.

25. The method of any one of claims 20-24, wherein the administration comprises an intravenous injection.

26. A bioassay for simultaneously measuring nanoparticle cell uptake and endosomal disruption, the bioassay comprising:
providing a nanoparticle comprising one or more fluorescent-labeled nucleic acids;
incubating the nanoparticle with Gal8-mRuby+ cells;
measuring nanoparticle uptake by quantifying fluorescent punta resulting from intracellular delivery of nanoparticles comprising the fluorescent-labeled nucleic acids; and
measuring endosomal disruption by quantifying mRuby fluorescent puncta resulting from Gal8-mRuby clustering at damaged endosomal membranes.

27. The bioassay of claim 26, wherein the fluorescent punta are quantified via images obtained by wide-field, epifluorescence microscopy.

28. A kit comprising a composition of any one of claims 1-15, a formulation of claim 16, or a nanoparticle of any one of claims 17-19.
